(19) United States
(12) Patent Application Publication
(10) Pub. No.: US 2025/0145934 A1
(43) Pub. Date: May 8, 2025

(54) FEEDBACK-CONTROLLED MEDIUM SCALE HOLLOW FIBER BIOREACTOR SYSTEM WITH INLINE SENSING AND AUTOMATED SAMPLING

(71) Applicant: GEORGIA TECH RESEARCH CORPORATION, Atlanta, GA (US)

(72) Inventors: Stephen BALAKIRSKY, Atlanta, GA (US); Annie BOWLES-WELCH, Atlanta,, GA (US); Walker Thomas BYRNES, Atlanta, GA (US); Bharat KANWAR, Atlanta, GA (US); Zhaonan LIU, Atlanta, GA (US); Krishnendu ROY, Marietta, GA (US); Bryan WANG, Atlanta, GA (US); Carolyn YEAGO, Atlanta, GA (US); Chuck ZHANG, Atlanta, GA (US)

(21) Appl. No.: 18/836,626

(22) PCT Filed: Feb. 7, 2023
(86) PCT No.: PCT/US2023/012492

§ 371 Date: Aug. 7, 2024

Related U.S. Application Data
(60) Provisional application No. 63/307,581, filed on Feb. 7, 2022.

(51) Int. Cl. C12M 1/36; C12M 1/12

(52) U.S. Cl. CPC C12M 41/48; C12M 25/10

(57) ABSTRACT

The present disclosure relates to improved biomanufacturing systems and methods with an ability to effectively control process variability to consistently generate high-quality cell-based therapies. In particular, disclosed herein is a using model-based control for determining process parameters in a bioreactor in real time. Additionally disclosed herein is a system for generating an AI digital twin model for a commercial-scale bioreactor.

### RELATED APPLICATION

This PCT application claims priority to, and the benefit of, U.S. Provisional Patent Application No. 63/307,581, filed Feb. 7, 2022, which is incorporated by reference herein in its entirety.

### GOVERNMENT LICENSE RIGHTS

This invention was made with government support under award no. GR10006329 awarded by the Food and Drug Administration. The government has certain rights in the invention.

### BACKGROUND

In recent years, cell-based therapies have emerged as effective weapons against various disease states. Biomanufacturing of cell therapies involves highly complex and labor-intensive processes, whereby the process parameters and biological variabilities can significantly influence product quality, reproducibility, and therapeutic efficacy of the products. Moreover, strict regulatory standards for quality create additional obstacles to generating therapeutically effective products. These issues are intensified in large-scale biomanufacturing operations, where minor stressors to a system can adversely affect production output and contribute to economic waste.

Current large-scale bioreactors, such as the Terumo Quantum, utilize off-line sampling and manual adjustment of feed rates in their expansion protocols and lack real-time monitoring to respond to variability in the biomanufacturing process. This framework can prompt delays in the correctional response, which lends to a decrease in yield and creates inconsistent product quality. Thus, there is a need for improved biomanufacturing systems and methods with an ability to effectively control process variability to predictably generate high-quality cell-based therapies.

### SUMMARY

The exemplary system and method employ its sensors and state estimation model to control for critical process parameters to maintain a more consistent growth environment for cells. Following the identification of critical quality attributes, what parameters or qualities of the cells are considered “ideal,” the system can be tuned, e.g., via a feedback controller, to maintain conditions during expansion which can yield cells that contain the desired qualities.

The exemplary system and method can be used for adherent cells, suspension cells, viral vectors, and exosomes.

In an aspect, disclosed herein is a method of using model-based control for determining process parameters in a bioreactor in real time (inline), the method comprising: a) providing a bioreactor comprising a culture chamber, wherein said culture chamber comprises live cells in culture media; b) providing two or more sensors for measuring two or more physical attributes of the culture media or the live cells therein; c) receiving, via a controller, sensor data from the two or more sensors operatively disposed on, or in a flow pathway of, the culture chamber; d) determining, via the controller, in real time, values associated with and derived from at least two physical attributes of the culture media or live cells therein using a model employing the sensor data from the two or more sensors; and e) adjusting, via the controller, according to a pre-defined control parameter, control of the culture media in the bioreactor using the determined values associated with and derived from physical attributes of the culture media or live cells therein.

In some embodiments, the live cells include immune cells, stem cells, stem-derived cells, or red blood cells. For example, in some embodiments, the immune cells comprise chimeric antigen receptor (CAR) T cells, natural killer cells (NKs), T-regulatory cells (T-regs), T cells, or tumor-infiltrating lymphocytes (TILs). In some embodiments wherein the live cells include stem cells, the stem cells comprise induced pluripotent stem cells (PSCs), mesenchymal stem cells (MSCs), or MSC- or PSC-derived cells.

According to some embodiments of the present method, the bioreactor comprises a stirred-tank, airlift, hollow-fiber, or rotary cell culture system (RCCS). In some embodiments, the bioreactor is a small-scale or medium-scale bioreactor.

In some embodiments, the two or more physical attributes comprise a critical quality attribute (CQA) and/or a critical process parameter (CPP). For example, in some examples, the two or more different physical attributes comprise dissolved oxygen, pH, glucose level, lactate concentration, temperature, conductivity, NO/NOx, volatile organic compounds (VOCs), ozone, chlorine, reduction-oxidation (redox) potential, agitation, inward gas flow, outward gas flow, pressure, vessel weight, metabolite concentrations, cytokine or growth factor concentrations, capacitance, or optical density. In some embodiments of the present method, the sensors provide data at discrete time intervals or continuously.

In some aspects, the method further includes: a feedback controller which automatically adjusts one or more physical parameters of the culture media based on the physical attributes of the culture media detected by the sensors. For example, in some aspects, one or more physical parameters are adjusted by the addition of a composition. In some embodiments, the amount or type of adjustment of the physical parameter is variable based on feedback received for that physical parameter.

Also disclosed herein is a system comprising components to perform the method described above.

In yet another aspect disclosed is a system for generating an AI digital twin model for a commercial-scale bioreactor, the system comprising: a) a bioreactor comprising a culture chamber, wherein said culture chamber comprises live cells in culture media; b) two or more sensors for measuring two or more different physical attributes of the culture media or the live cells therein; and c) a computing device communicatively coupled to the two or more sensors, the computing device comprising i) a processor; ii) a memory; and iii) a communication module; the processor communicatively coupled to the memory and the communication module; the processor configured to receive sensor data from the two or more sensors to train an AI model configured to determine values associated with and derived from physical attributes of the culture media or live cells therein using the sensor data from the two or more sensors, wherein the trained AI model is subsequently employed in controls of the commercial-scale bioreactor to determine values associated with and derived from physical attributes of the culture media or live cells therein using sensor data from sensors of the commercial-scale bioreactor.

In some embodiments, the live cells comprise immune cells, stem cells, stem-derived cells, or red blood cells. In some embodiments, the immune cells comprise chimeric antigen receptor (CAR) T cells, natural killer cells (NKs), T-regulatory cells (T-regs), T cells, or tumor-infiltrating lymphocytes (TILs). In some examples, the stem cells comprise induced pluripotent stem cells (PSCs), mesenchymal stem cells (MSCs), or MSC- or PSC-derived cells.

In some embodiments, the bioreactor comprises a stirred-tank, airlift, hollow-fiber, or rotary cell culture system (RCCS). In some embodiments, the bioreactor is a small-scale or medium-scale bioreactor.

In some embodiments, said physical attributes include a critical quality attribute (CQA) and/or a critical process parameter (CPP). In some further embodiments, the two or more different physical attributes comprise dissolved oxygen, pH, glucose level, lactate concentration, temperature, conductivity, NO/NOx, volatile organic compounds (VOCs), ozone, chlorine, reduction-oxidation (redox) potential, agitation, inward gas flow, outward gas flow, pressure, vessel weight, metabolite concentrations, cytokine or growth factor concentrations, capacitance, or optical density. In some examples of the presently disclosed system, the sensors provide data at discrete time intervals or continuously.

In some further examples, the system comprises a feedback control component that automatically adjusts one or more physical parameters of the culture media based on the physical attributes of the culture media detected by the sensors.

### BRIEF DESCRIPTION OF THE DRAWINGS

The skilled person in the art will understand that the drawings described below are for illustration purposes only.

FIG. 1 shows a schematic diagram of exemplary systems for generating an AI digital twin model for a commercial-scale bioreactor according to the present disclosure.

FIGS. 2A-2B each show exemplary methods using model-based control for determining process parameters in a bioreactor.

FIG. 3 shows a fiberCell bioreactor to which a model can be generated.

FIGS. 4A-4H illustrate: FIG. 4A. Simplified 1D convection-diffusion model for hollow-fiber bioreactors, which also includes novel dosing solution, inlet and recirculation flowrates, and input/output combined glucose and lactate sensors. FIG. 4B. Simplified exponential cell-growth model and fixed glucose/lactate rates. FIG. 4C. Experimental glucose step response to determine optimal fluidic and membrane parameters for the model. FIG. 4D. Resulting model parameters from the above model validation including diffusion constants for glucose and lactate, fiber membrane constants, and cell growth and consumption constants. FIG. 4E. Overall Control Architecture Design (LC). The EKF Observer uses the model and the sensor readings to update concentrations of unmeasured states inside the bioreactor. The controller computes optimal flowrates to correct linear errors while the feedforward model corrects for exponential cell-growth. FIG. 4F. Glucose response using this protocol (LC) vs the Quantum Protocol (QP).

FIG. 4G. Lactate response for LC vs. QP. FIG. 4H. Simulated study results. Significantly improved glucose and lactate errors and reduced lactate spikes. Both LC and QP simulated with the same parameters and media limitations.

FIGS. 5A-5C show results from MSC expansion carried out on a scaled-down hollow-fiber bioreactor model. FIG. 5A. Total cell count before and after expansion, with a fold expansion of 3.4. FIG. 5B. IDO activity diagram. IDO activity indicates the ability of the MSC for T-cell suppression which is an important indication of immunomodulatory function. FIG. 5C. Reduced IDO activity of cells after expansion.

FIGS. 6A-6C show a schematic for a cell density sensing system (FIG. 6A) and the bioreactor integration (FIG. 6B). Two-electrode cross-membrane design, where AC currents flow across the fiber walls. Expendable EC probes are inserted in the cartridge, while the IC probe is inserted at the IC inlet/outlet. The measurement system automatically picks up impedance spectra between several EC probes and a common IC probe. The resulting figure (FIG. 6C) shows frequency-implicit impedance spectra and fitting results collected from 4 probes without cells seeded. Sterile installation of the probes is still in progress. Expected correlation between cell density on the fiber walls and capacitance.

FIG. 7 shows an example bioreactor design for the expansion of MSCs. The models include the hollow fiber model and the vertical wheel model.

FIG. 8 shows an optimization approach taken employing fractional factorial design and response surface methodology.

FIG. 9 shows an example processing that can be employed by the system.

FIG. 10 shows an automation function that can create automated and responsive unit operations for maintaining culture efficiency, increasing culture reproducibility, and reducing labor requirements. The automated controller is able to eliminate metabolite spikes in media profile, keeping glucose level high and lactate level low, potentially creating high-quality MSC products.

FIG. 11 shows an experimental setup of surrogate bioreactor systems.

FIG. 12 shows that the vertical wheel bioreactor can be optimized to be a suitable culturing method for CT-MSCs in a serum-free condition.

FIG. 13 shows the results of the vertical wheel bioreactor between optimized and unoptimized configurations.

FIG. 14 shows that a customized cell density sensor can pick up responses with cells attaching to fibers.

FIG. 15 depicts plots illustration that the automation approach can reduce process disturbances; experimental results match simulation results.

FIG. 16 shows a hollow-fiber bioreactor that includes a novel dosing solution, inlet and recirculation flowrates, and input/output combined glucose and lactate sensors.

FIGS. 17A-17G show (FIG. 17A) 1D convection-diffusion model for hollow-fiber bioreactors, including novel dosing solution, inlet and recirculation flowrates, and input/output combined glucose and lactate sensors. (FIG. 17B) Time-varying cell growth and death parameters were added to the model. (FIG. 17C) Overall Control Architecture Design with EKF observer to estimate unmeasurable states/model parameters and an optimal controller to regulate Glucose and Lactate. (FIG. 17D) Results from baseline cell expansion and process-controlled cell expansion. Baseline vs. Controlled glucose (FIG. 17E) and lactate (FIG. 17F) concentrations. (FIG. 17G) Controlled expansion flowrates.

FIGS. 18A-18E show FIG. 18A. Timeline for FiberCell MSC expansion process. FIG. 18B. One FiberCell expansion yielded 56 million cells, while one T flask (225 cm2) yielded 12 million cells. FIG. 18C. IDO activity levels correlate with MSC immunosuppressive activities. Based on the results, no significant differences were observed between cells expanded from T flask and FiberCell. FIG. 18D. MSCs suppress TNF secretion by macrophages when co-cultured. Results showed no significant differences between cells expanded from the T flask and FiberCell. FIG. 18E. MSCs exert immunosuppressive functions by suppressing T cell proliferation. Results show cells expanded from T flask and FiberCell have similar suppression abilities.

FIGS. 19A-19C show FIG. 19A. Schematic for cell density sensing system and bioreactor integration. EC probes are inserted in the cartridge, while the IC probe is inserted at the IC inlet/outlet. FIG. 19B. Probes detect the hollow fiber wall's impedance, which reflects the cell density. FIG. 19C. Parameter A can serve as an indicator of cell density in the hollow fibers.

FIG. 20 shows a control architecture with an exemplary fluid model, cell model, observer, and controller.

FIG. 21 shows an overview of the bioreactor model with updated fluid dynamics and cell dynamics.

FIG. 22 shows an example of the bioreactor setup used for expansions.

FIG. 23 shows model validation with a post-expansion fitted C_{g} model.

FIG. 24 shows model validation of waste cumulative lactate with post-expansion fitted C_{g} model.

FIG. 25 shows model validation of the cumulative lactate dynamic model and C_{g} sensor model.

FIGS. 26A-26B show simulated input flowrates comparing baseline, controlled, and smooth, controlled protocols for (FIG. 26A) input media flowrates and (FIG. 26B) input concentration flowrates.

FIGS. 27A-27D show bioreactor expansion with hMSCs using an updated observer model for fluidic and estimated cell parameter states and updated LQR controller for (FIG. 27A) Glucose measurements compared to observer model (FIG. 27B) Lactate measurements compared to observer model, (FIG. 27C) Estimated cell number compared to final yield, and (FIG. 27D) Estimated cell growth rate.

FIGS. 28A-28C depict the overall automation design for the vertical wheel bioreactor with media and dosing feed lines, waste and bypass loop for sensing and sampling, circulation loops for continuous imaging, and flush line to remove cells after imaging (FIG. 28A). Schematic for hMSC cell growth experiment (FIG. 28B) and the associated physical setup in the incubator (FIG. 28C).

FIGS. 29A-29B show simulated control performance with 100 mL input fresh media under exponential cell growth for glucose (FIG. 29A) and lactate (FIG. 29B). Microcarrier imaging during cell expansion and after harvest (FIG. 29C). Preliminary expansion results measuring cell yield, fold expansion and cell viability for standard vertical wheel, the vertical wheel with imaging, and control flask

### DETAILED DESCRIPTION

To facilitate an understanding of the principles and features of various embodiments of the present invention, they are explained hereinafter with reference to their implementation in illustrative embodiments.

### Definitions

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising,” particularly in a description of components of a composition or in a description of elements of a device, can be exchanged with “consisting essentially of” or “consisting of.” The invention illustratively described herein suitably may be practiced in the absence of any element or elements, limitation, or limitations which is not specifically disclosed herein. In each instance herein, any of the terms “comprising,” “consisting essentially of,” and “consisting of” may be replaced with either of the other two terms.

One of ordinary skill in the art will appreciate that methods, device configurations and combinations, device elements, processes, organisms, cells, and media other than those specifically exemplified can be employed in the practice of the invention without resorting to undue experimentation. It will be particularly appreciated by those of ordinary skill in the art that bioreactor styles, bioreactor wall locations, bioreactor cover designs, bioreactor filter locations, sealing methods, porous materials, xerogels, aerogels, sol-gel glasses, cells, organisms, cellular components, products, hydrogen production methods, hydrogen-producing cells and organisms, culturing methods, culture media, inert gases, gases, culture circulation methods, bioreactor sanitizing and sterilizing methods, and gas sparge methods, other than those specifically disclosed herein are available in the art and can be readily employed in the practice of this invention All art-known functional equivalents of any such device element and combinations, methods, materials as well as cells and organisms are intended to be encompassed within the scope of this invention.

All patents and publications mentioned in the specification are indicative of the levels of skill of those skilled in the art to which the invention pertains. References cited herein are incorporated by reference herein in their entirety to indicate the state of the art as of their filing date, and it is intended that this information can be employed herein, if needed, to exclude specific embodiments that are in the prior art.

One skilled in the art will readily appreciate that the present invention is well adapted to carry out the objects and obtain the ends and advantages mentioned, as well as those inherent therein. The devices, device elements, methods, and materials described herein as presently representative of preferred embodiments are exemplary and are not intended as limitations on the scope of the invention. Changes therein and other uses will occur to those skilled in the art and are intended to be encompassed within this invention.

As used herein, “about” refers to a value that is 10% more or less than a stated value.

As used herein, “agitate” refers to perturb such that the liquid phase of the culture is in dynamic interaction with the gas phase above the culture. Agitate may refer to a motion such as shaking, stirring, rocking, orbital shaking, rolling, figure-eight shaking or any means to make the liquid phase non-static and increase the diffusion of gases in and out of the liquid phase.

As used herein, “gas” refers to a pure gas or mixture of gases which may include nitrogen, oxygen, and carbon dioxide. Typically, nitrogen is present in an amount of about 60 to 90% of the total gas concentration; oxygen is present in an amount of about 10 to 40% of the total gas concentration, and carbon dioxide is present in an amount of about 0 to 50% of the total gas concentration.

As used herein, “cell culture” refers to a liquid preparation containing eukaryotic cells in a liquid medium containing buffering agents and nutrients required for the growth and/or maintenance of viable cells.

As used herein, “concentration of dissolved CO₂” is expressed by the relative measure of partial pressure of CO₂ in mmHg. Therefore, the partial pressure of dissolved CO₂ is used as a reflection of the concentration of dissolved CO₂.

“DO” refers to dissolved oxygen.

“Critical Quality Attribute” (CQA) refers to a physical, chemical, biological, or microbiological property or characteristic that should be within an appropriate limit, range, or distribution to ensure the desired product quality.

“Critical Process Parameter” (CPP) refers to a process parameter whose variability has an impact on a critical quality attribute and, therefore, should be monitored or controlled to ensure the process obtains the desired quality.

“Key Performance Indicator” (KPI) refers to a metric for the status of each production step. KPIs are related to CQAs and therefore influenced, as well, by the CPPs. As the CPPs remain within the pre-defined limits, the KPIs should indicate that each production step proceeds accordingly, resulting, in the end, in a product having its CQAs within the appropriate limits, too. CQAs are still difficult to measure directly in product.

As used herein, “dynamic interface” refers to an enhanced active exchange of gases between a liquid phase and gas phase provided by the agitation of the cell culture.

As used herein, “eukaryotic cells” refers to animal cells which may be invertebrate or vertebrate cells.

As used herein, “bioreactor” refers to a vessel, including an open or closed vessel, for culturing one or more cells or organisms or for maintaining or producing cellular components, including recombinant proteins. In some embodiments, a bioreactor is used for the production of a therapeutic protein (e.g., a recombinant protein) by cultured cells. In some embodiments, bioreactors are made of corrosion-resistant alloys, such as stainless steel (e.g., grade-316L stainless steel). However, in some embodiments, a bioreactor may be made of glass, ceramics, plastic, or any number of materials or combinations thereof. In some embodiments, a bioreactor is configured with one or more supply lines for supplying nutrients, glucose, O₂, CO₂, and other components to the bioreactor. In some embodiments, a bioreactor is configured with one or more output lines for removing waste or other components from the bioreactors. In some embodiments, a bioreactor is configured with one or more spargers for bubbling a gas (e.g., O₂, CO₂) through a culture medium. In some embodiments, a bioreactor comprises one or more agitators or mixes for mixing a culture medium. In some embodiments, a bioreactor comprises one or more heating elements and one or more thermocouples configured to permit the temperature of the bioreactor culture to be controlled. In some embodiments, a bioreactor is configured with a spectroscopic instrument configured for obtaining spectroscopic measurements on a culture.

### Methods and Systems

Specifically, disclosed herein is a method of using a model-based control for determining process parameters in a bioreactor in real time (inline), the method comprising: a) providing a bioreactor comprising a culture chamber, wherein said culture chamber comprises live cells in culture media; b) providing two or more sensors for measuring two or more physical attributes of the culture media or the live cells therein; c) receiving, via a controller, sensor data from the two or more sensors operatively disposed on, or in a flow pathway of, the culture chamber; d) determining, via the controller, in real time, values associated with and derived from the at least two attributes of the culture media or live cells therein using a model employing the sensor data from the two or more sensors; and e) adjusting, via the controller, according to a pre-defined control parameter, control of the culture media in the bioreactor using the determined values associated with and derived from physical attributes of the culture media or live cells therein.

Also disclosed herein is a system for generating an AI digital twin model for a commercial-scale bioreactor, the system comprising: a) a bioreactor comprising a culture chamber, wherein said culture chamber comprises live cells in culture media; b) two or more sensors for measuring two or more different physical attributes of the culture media or the live cells therein; and c) a computing device communicatively coupled to the two or more sensors, the computing device comprising i) a processor; ii) a memory; and iii) a communication module; the processor communicatively coupled to the memory and the communication module; the processor 122 configured to receive sensor data from the two or more sensors to train an AI model configured to determine values associated with and derived from physical attributes of the culture media or live cells therein using the sensor data from the two or more sensors, wherein the trained AI model is subsequently employed in controls of the commercial-scale bioreactor to determine values associated with and derived from physical attributes of the culture media or live cells therein using sensor data from sensors of the commercial-scale bioreactor.

Any type of bioreactor can be used with the methods and systems disclosed herein. For example, the bioreactor can be any stirred-tank, airlift, hollow-fiber, or rotary cell culture system (RCCS). The bioreactor can be small-scale, mid-scale, or large-scale.

A variety of cells can be grown in a bioreactor. Such cells include, but are not limited to, immune cells, stem cells, stem-derived cells, or red blood cells. Examples of immune cells are chimeric antigen receptor (CAR) T cells, natural killer cells (NKs), T-regulatory cells (T-regs), T cells, or tumor-infiltrating lymphocytes (TILs). Examples of stem cells comprise induced pluripotent stem cells (PSCs), mesenchymal stem cells (MSCs), or MSC- or PSC-derived cells.

The physical attribute measured can be a variety of things. For example, it can be a critical quality attribute (CQA) and/or a critical process parameter (CPP). Examples of CQAs and CPPs that can be monitored include, but are not limited to, dissolved oxygen, pH, glucose level, temperature, conductivity, NO/NOx, volatile organic compounds (VOCs), ozone, chlorine, reduction-oxidation (redox) potential, agitation, inward gas flow, outward gas flow, pressure, impedance, resistance, vessel weight, metabolite concentrations, cytokine or growth factor concentrations, capacitance, cell size, cell dynamics, distribution of cell size or dynamics, or optical density. For other examples, see the white paper by Hamilton® entitled “Biopharma PAT Quality Attributes, Critical Process Parameters, and Key Performance Indicators at the Bioreactor” (May 2018), herein incorporated by reference in its entirety.

The sensors found in the bioreactors can be any type of sensor that can detect any type of parameter in either the cells or the culture thereof. For example, “Integrated multi-sensor system for parallel in-situ monitoring of cell nutrients, metabolites, cell density and pH in biotechnological processes,” Stefan Mrossa, Tom Zimmermanna, Nadine Winkinb, Michael Kraftc, Holger Vogta, Sensors and Actuators B 236(2016) 937-946, herein incorporated by reference in its entirety for its teaching concerning sensors, describes various types of sensors which can be used with the methods and system of the present invention.

The bioreactor can include various types and numbers of sensors, such as chemical sensors, electrical sensors, thermal sensors, etc. as would be understood by one having ordinary skill in the art. The sensor(s) can be configured to detect and/or observe one or more phenomena and/or collect data relating to the CQAs and CPPs discussed above. For example, the sensors in one embodiment may include one or more of electrical sensors configured to detect one or more electrical conditions, such as an electrical potential across a cell or population of cells; optical sensors configured to detect one or more optical phenomena such as an amount of light present/emitted within the reservoir (e.g. light of a particular wavelength being emitted by one or more cells, such as in a fluorescent in-situ hybridization experiment); chemical sensors configured to detect chemical conditions in the reservoir such as presence and/or concentration of a target compound or compounds (including biological molecules such as the indicators, markers, an neurotransmitters described above, gases such as carbon monoxide, carbon dioxide, oxygen, nitrogen, etc. as would be understood by one having ordinary skill in the art), pH, etc.; mechanical sensors configured to detect one or more mechanical forces acting on contents of the reservoir (e.g. physical strain on cells, e.g. shear stress, surface tension, etc.), thermal sensors configured to detect an environmental temperature in one or more regions of the bioreactor (e.g. in the enclosure, in the walls, in the reservoir, etc.). Any combination of the aforementioned sensors and/or sensor functionalities may be utilized in various embodiments. The sensors can provide data at discrete time intervals or continuously.

In some embodiments, disclosed are methods for evaluating a culture medium or the cells thereof. For example, a culture component level in a culture medium can be evaluated. In some embodiments, the analysis of a culture medium comprises determining the presence of one or more culture components in a biological sample.

In some embodiments, the analysis of a biological sample comprises evaluating the level of a culture component. It should be appreciated that the methods provided herein allow for the analysis of a wide variety of culture media and biological samples. Culture media and biological samples, as used herein, refer to media and samples that include one or more components (e.g., glucose) of a biological production process. In some embodiments, evaluating a culture medium includes evaluating the presence of one or more components (culture components) in a biological sample or culture medium. In some embodiments, evaluating a culture medium includes evaluating the level of one or more components in a biological sample. In some embodiments, the presence or level of one or more culture components can be correlated to the quality of the sample and/or the progress of a particular biological manufacturing process.

In some embodiments, the level of a component during the biological production process can be used to monitor the progress of the biological production process. Thus, for instance, if glucose is consumed during a biological production process, the presence of the same level of glucose during the progression of the biological production process as at the beginning of the biological production process is a sign that the bioprocess is not proceeding as desired. In addition, the presence of a new component can be a sign that the biological production process is proceeding in some embodiments, or not proceeding in other embodiments as planned. Thus, a biological production process may be monitored for the occurrence of desired product or indicator that the biological production process is progressing as desired. On the other hand, the presence of a particular metabolite may be a sign that cells in the biological production process are not generating the desired product but, for instance, are merely proliferating. Thus, determining the presence of one or more components in a biological sample is a way of evaluating the sample and predicting the successfulness (e.g., yield) of a biological production process.

It should be appreciated that the component analysis can also be expanded to multiple components. Thus, for instance, a biological production process may require a particular ratio of glucose to glutamate to proceed optimally. A sample may be monitored prior to or throughout the reaction for this relationship, and the conditions may be adjusted if the observed ratio deviates from the desired ratio.

In a preferred embodiment, the method described herein can comprise the additional component of a feedback control, which automatically adjusts one or more physical parameters/attributes of the culture media based on the physical attributes/parameters of the culture media or cells, as detected by the sensors. Such feedback loops are known to those of skill in the art and can be integrated with the systems disclosed herein. For example, if a sensor determines that glucose levels are sub-optimal, the feedback component of the system can sense this and provide feedback to an automated controller, which can then provide glucose at a level and for a length of time as appropriate. The system disclosed herein can allow this to happen inline using the twin model system described herein. Therefore, one or more physical parameters can be adjusted by the addition of a composition. The amount or type of adjustment of the physical parameter is variable based on feedback received for that physical parameter.

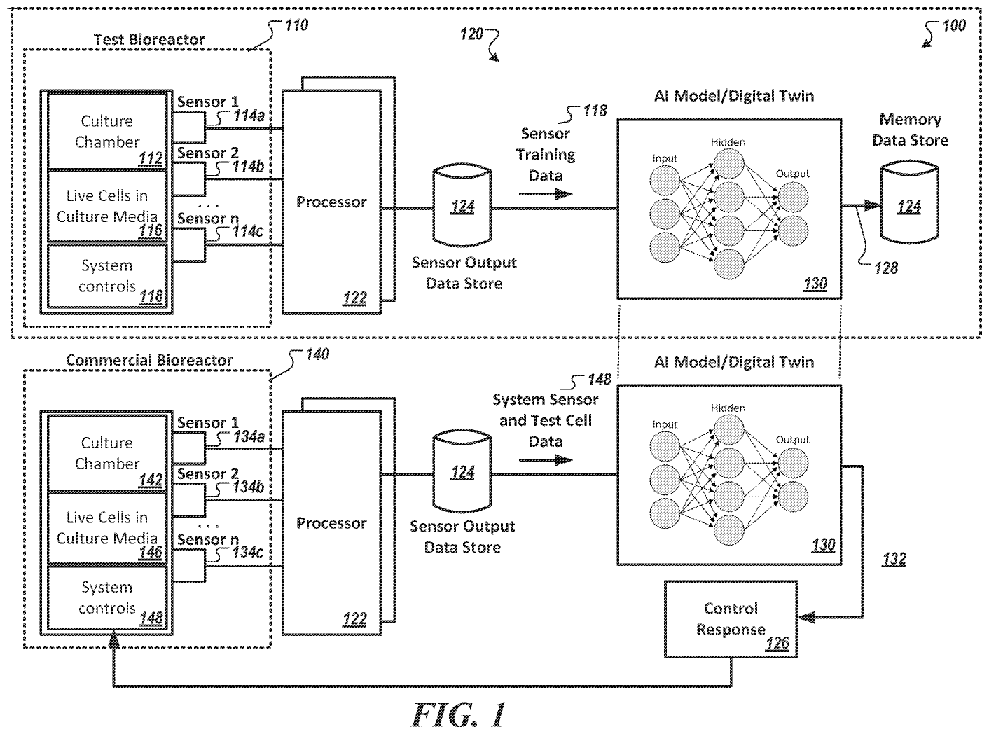

The AI model 130 shown in FIG. 1 can predict, or estimate, culture growth rate, cell number, or other cell or culture characteristics based on measured parameters, as discussed above.

Machine Learning. In addition to the machine learning features described above, the system and method can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan h, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by down sampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

For example, PCT Application No. WO2019/210405, herein incorporated by reference in its entirety, teaches computerized control of a bioreactor. The above-described methods may be based on the real-time monitoring and processing of multiple bioreactor sensor readings in order to determine, in real-time, predicted cell biomass production, or may be based on the monitoring of multiple bioreactor sensor readings at discrete time intervals (which may be preconfigured), or on-demand based on user input. It will be appreciated that in order to determine or predict a parameter, substantially in real-time upon user request (or upon receipt (at discrete intervals or continuously) of sensor data, although in certain embodiments, the determination or prediction of an approximate amount of product may not be performed in real-time upon receipt of sensor data), the above-described methods are required to be computer-implemented. As used herein, the term “real-time” includes “substantially real-time”, to account for lags in computer processing, digital read/write operations, and communications, transmissions, as would be known to the skilled person in the art.

The processor, as described herein, may make the determination in real-time (or not in real-time) and recursively based on continuous inputs from the sensors. In a further embodiment, the bioreactor may include a (or there may be a separate) feedback controller communicatively coupled to the processor and to one or more feed input ports (or output port(s)) of the bioreactor, and which causes the feed to be provided to (or substance to be removed from) the bioreactor based parameter predictions, and said feedback controller may receive and provide feedback control of bioreactor input(s) (or output(s)) based on real-time (or not real-time) predictions. In yet further embodiments, the processor and feedback controller/processor may be housed in the same physical unit, or may comprise the same unit. In yet other embodiments, any controllers or processors utilized for the described methods and systems may be remotely located from the bioreactor. Communications between the controllers and bioreactor may be via wired or wireless connection, using transmitters, receivers, and/or transceivers. Further aspects of systems for carrying out the methods herein are described below.

In addition to the bioreactors and sensors described herein, the system for carrying out the presently described methods may further comprise a computing device comprising a processor, a memory, an input device, and a communication module. The computing device may further comprise receivers, transmitters and/or transceivers. The processor may be communicatively coupled to the memory, the input device, the communication module, and/or any receivers, transmitters and/or transceivers, and as described herein, the computing device may be remotely located from or co-located with the bioreactor, and communicatively coupled to the sensors. All processors described herein may be configured to carry out any or all method steps suitable for computer implementation. For example, in an embodiment, the processor may be configured to: receive (e.g., via a receiver or the communication module) sensor data from the two or more sensors; and determine, based on a predetermined or recursively adapted or recursive algorithm stored in the memory that correlates the sensor data of two or more different physical attributes or conditions (such as those described above) and product production by independent consideration of the sensor data of the two or more different physical attributes or by multivariate consideration of the sensor data of the two or more different physical attributes, an approximate amount of the product.

In embodiments involving the determining and/or adjustment of the rate of process input (e.g., feed) supply to the bioreactor, the system may further comprise a feedback controller for carrying out the adjustment of the rate of the process input (e.g., feed) supply, as discussed above. The feedback controller may be communicatively coupled to the computing device and the bioreactor, such as to a feed input port(s) (or output port(s)) of the bioreactor. Upon receipt from the communication module of the computing device of the determined adjusted rate of process input (e.g., feed) supply, as determined by the processor in accordance with methods described herein, the feedback controller may adjust the rate of the supply of the process input (e.g., feed) to the bioreactor to the rate of supply of the process input (e.g., feed) determined by the processor, such as by opening, closing, or otherwise adjusting the rate of flow of the process input (e.g., feed) through the input or output port(s) of the bioreactor.

The processor of the described system, for processing information associated with the process(es) or method(s) described herein, may be, for example, any computer processor is known in the art capable of performing calculations and directing functions for interpreting and/or performing input, output, calculation, and display of data in accordance with the disclosed methods. The processor may comprise any type of processing unit, such as typical computer processor(s), controller(s), microcontroller(s), microprocessor s), and/or programmable logic controllers (PLCs). The information to be processed by the processor may include, for example, information contained in analog or digital signals and/or translated signals and/or information contained in a data storage. Processing of the information may involve, for example, performing calculations on received signals such as, but not limited to, vector analysis, picture identification, pattern recognition, frequency analysis/Fourier transforms, numerical computations, machine learning, or, as described herein, applying predetermined or recursively fit correlative algorithms to received sensor data. In some embodiments, the system comprises more than one processor, and the reference herein to “processor” includes reference to multiple processors and vice versa.

In an embodiment, the processor is in communication with the two or more sensors of the bioreactor. In another embodiment, the processor may also be in communication with data storage(s), and, optionally, display(s). The components of the system, such as the bioreactor, sensors, computing device(s), processor(s), feedback controller(s), data storage(s), and/or display(s), and any other components of system, bioreactor or computing device, may communicate using any electronic wired or wireless means or protocols for communication known in the art, including but not limited to Ethernet™, Bluetooth™, WiFi™, infrared, near-field communications (NFC), radio-frequency identification (RFID), WiMAX™ (fixed or mobile), cellular communications protocols such as GSM, EDGE, GPRS, CDMA, EMTS, LTE, LTE-A, IMS, and any other cellular communications protocols including, but not limited to, up to and including 5G protocols as established under the 3 GPP, for example, and any other communications protocols suitable for the method(s) and system(s) described herein, including any proprietary protocols. Components of the system may exist on the same network or on separate networks, and the network(s) may include any type of network suitable for the system(s) and method(s) described herein, including but not limited to wired or wireless personal area networks (PANs), local area networks (LANs), mesh or ad hoc networks, wide area networks (WANs), metropolitan area networks (MANs), virtual private networks (VPNs), and any other suitable network type, as well as any suitable network configuration or topology (e.g., token ring, star, bus, mesh, tree, etc.). The presently described system(s) further includes any components necessary to effect the communication and/or network type employed, such as wireless or wired routers and access points.

The presently described methods and systems may be implemented on a secure network to which access may be limited to authorized users by any known means and which may be protected by known security measures, such as by the use of firewalls. In some embodiments, authentication may be required before granting access to authorized users, such as where autologous cell manufacturing and/or patient data is involved and/or where compliance with government regulations is mandated (such as Title 21 of the U.S. Code of Federal Regulations). Such authentication may be implemented for any one or more of the system components, such as for access to a computing device or machine housing the processor, access to data storage, access to a database of the data storage, access to any of the sensors or sensor readings of the bioreactor, access to a graphical user interface (GUI) of the system, access to the bioreactor, etc. Security of the presently described methods and systems may be further provided for by encrypting communications among system components by any means or protocols known to persons skilled in the art, such as Internet Protocol Security (IPSec), Transport Layer Security (TLS), Secure Sockets Layer (SSL), etc., in order to reduce the potential for the tampering with or corruption of the preprogrammed correlative algorithm(s), for example

The presently described system may also include a data storage for storing information associated with the described methods. The data storage may include, for example, various types of local or remote memory devices such as a hard disk or hard drive (of any type, including electromechanical magnetic disks and solid-state disks), a memory chip, including, e.g., random-access memory (RAM) and/or read-only memory (ROM), flash memory, optical memory such as CD(s) and DVD(s), floppy disks, and any other form of optical, physical, electronic, and/or magnetic memory devices in or on which information may be stored. The data storage may comprise non-volatile memory. In some embodiments, the data storage may only be accessed via secure data transfer, which may be accomplished using one or more known server platforms and security protocols. The information to be stored in the data storage may comprise, for example, one or more predetermined algorithms for correlating sensor inputs to various outputs, such as cell biomass production, and records of such predicted determinations along with associated actions (such as feedings) and/or associated timestamps, unique identifiers, authorized users and associated authentication information for use in authenticating users for authorized access to the data storage or any other system component, and any other pertinent information. In operation, the data storage is in communication with the processor.

The system may also include a display (which may be co-located with the processor, e.g., where the processor and display are part of a computer or server used for carrying out the method steps described herein) for visually presenting information associated with the described methods. The display may comprise, for example, a computer monitor (e.g., LCD, a CRT monitor, a projection (e.g., heads-up display (HUD) laser), etc. In some embodiments, the visual display may comprise, for example, that of a mobile device such as a tablet computer, cellular phone, smartphone, personal digital assistant (PDA), personal computer (PC), laptop computer, augmented reality display (e.g., Google™ Glass™ or Microsoft™ HoloLens™), etc. The information presented on the display may be as shown in FIG. 9, for example, and/or may include any other information collected in the course of carrying out the methods described herein, prompts for information entry associated with one or more steps of the described methods, and/or any predetermined formulae or algorithms, as previously described. The display may also be capable of receiving input (such as, e.g., where the display includes a touch-screen and is capable of receiving touch input and accordingly transmitting information to the processor).

It will be appreciated that components of the described system, such as a computer or machine housing the processor(s), include components known in the art that is required for their operation, such as a power supply, a network interface (such as a network interface card), network connectivity components (e.g., a modem, Ethernet cards, USB interface cards, FDDI cards, WLAN cards, etc.), a receiver, a transmitter, local memory, e.g., RAM, ROM, flash memory, cache or buffer memory, and/or other types of memory as previously described, a processing unit which may be in communication with input/output (EO) devices, and all required circuitry, including bus(es). The bioreactor sensors 1404 may also include components specific to the sensor type, as would be known to the skilled person in the art. A computing device 1450, such as a computer or machine, housing the processor(s) may also include, for example, memory (e.g., hard disk storage, RAM, ROM, flash memory, cache or buffer memory, and/or other types of memory as previously described), attached input device(s) (e.g., a mouse, keyboard, microphone, etc.), attached output device(s) (e.g., a display monitor), and local memory for the processor(s) (e.g., registers, cached RAM, such as L1 cache, L2 cache, etc.). Depending on the system component, other components may also be present (e.g., a device in communication with a cellular network may include an antenna, etc.), and it will be appreciated that such components would be known to the skilled person in the art.

The described systems may comprise one or more redundant sensors for each of the two or more sensor types in the bioreactor to provide further data for determining the correlation and thus to further improve the predictive accuracy of cell biomass production based on the sensor readings (i.e., while each of the two or more sensor types in the presently described methods and systems are different types of sensors, each different type of sensor may have associated redundant sensors of the same type). Redundant sensor(s) for each of the two or more sensor types in the bioreactor may also be used in larger bioreactors to ensure the system is homogenous. Where, e.g., the sensor information is consistent among the redundant sensors, it may be stored in the data storage (e.g., in a database thereof), and where the information captured is not consistent among the redundant sensors, the respective sensor readings may be averaged or ignored altogether, or one of the redundant sensor readings, if not generally in line with the trajectory of the other received sensor data, may be discarded as an outlier, as previously described. Redundancy of the data storage and/or a database thereof is expected to help ensure the persistence of stored data and reduce the risk of data loss.

In some embodiments, the method(s) provided herein may be implemented using computer-readable and executable instructions, as described above, for example. Accordingly, another aspect provided herein is a tangible, non-transitory computer-readable medium (i.e., a medium which does not comprise only a transitory propagating signal per se\ such as memory, comprising or having stored thereon or therein computer-executable instructions associated with the method(s) described herein, such as a local or remote hard disk or hard drive (of any type, including electromechanical magnetic disks and solid-state disks), a memory chip, including, e.g., random-access memory (RAM) and/or read-only memory (ROM), cache(s), buffer(s), flash memory, optical memory such as CD(s) and DVD(s), floppy disks, and any other form of a storage medium in or on which information may be stored in a volatile or non-volatile manner, for any duration, included permanently or for brief instances. Such computer-executable instructions, if executed by a processor (e.g., of a computing device 1450, such as a computer housing the processor(s) described herein), cause the processor(s), and/or the computing device, computer or machine, to perform any of the method steps described herein that are suitable for computer implementation. Different implementations of the disclosed method(s) may involve performing some or all the steps described herein in different orders or some or all of the steps substantially in parallel. The functions or method steps may be implemented in a variety of programming languages, and such code or computer-readable or executable instructions may be stored or adapted for storage in one or more machine-readable media, such as described above, which may be accessed by a processor-based system to execute the stored code or computer-readable or executable instructions.

It will be appreciated that any method step, module, component, or system described herein that is suitable for computer implementation or required to be computer-implemented so as to effect real-time or substantially real-time execution may be implemented using computer readable/executable instructions or operations that may be stored or otherwise held by computer-readable media, as described herein.

### Example System

FIG. 1 shows schematic diagrams of a system 100 for generating an AI digital twin model for a commercial-scale bioreactor 140.

In FIG. 1, the system 100 includes a test bioreactor 110 comprising a culture chamber 112, wherein said culture chamber 112 comprises live cells in culture media 116; two or more sensors 114 (shown as 114a, 114b, 114c) for measuring two or more different physical attributes of the culture media or the live cells therein 116. The system 100 shown in FIG. 1 further includes system controls 118, which can regulate a physical attribute of the test bioreactor 110. System 100 also includes a computing device 120 communicatively coupled to the two or more sensors 114. The computing device 120 includes: i) a processor 122; ii) a memory 124; and iii) a communication module 126; the processor 122 communicatively coupled to the memory 124 and the communication module 126; the processor 122 configured to receive sensor data 118 from the two or more sensors 114 to train an AI model 130 configured to determine values associated with and derived from physical attributes 128 of the culture media or live cells therein 116 using the sensor data 118 from the two or more sensors 114. The trained AI model 130 of system 100 is subsequently used to determine values associated with and derived from attributes 132 of the culture media or live cells therein 146 using sensor data 148 obtained from sensors 144 (shown as 144a, 144b, 144c) of the commercial-scale bioreactor 140. The values associated with and derived from attributes 132 are then used to determine a predictive control response 126 of the commercial-scale bioreactor 140, which can modify one or more physical parameters of the culture media 116 via system controls 148.

In some embodiments, the live cells comprise immune cells, stem cells, stem-derived cells, or red blood cells. In some embodiments, the immune cells comprise chimeric antigen receptor (CAR) T cells, natural killer cells (NKs), T-regulatory cells (T-regs), T cells, or tumor infiltrating lymphocytes (TILs). In some examples, the stem cells comprise induced pluripotent stem cells (PSCs), mesenchymal stem cells (MSCs), or MSC- or PSC-derived cells.

In some embodiments, the bioreactor (e.g., the test bioreactor and/or the commercial bioreactor) comprises a stirred-tank, airlift, hollow-fiber, or rotary cell culture system (RCCS). In some embodiments, the test bioreactor is a small-scale or medium-scale bioreactor.

In some embodiments, said physical attributes include a critical quality attribute (CQA) and/or a critical process parameter (CPP). In some further embodiments, the two or more different physical attributes comprise dissolved oxygen, pH, glucose level, temperature, conductivity, NO/NOx, volatile organic compounds (VOCs), ozone, chlorine, reduction-oxidation (redox) potential, agitation, inward gas flow, outward gas flow, pressure, vessel weight, metabolite concentrations, cytokine or growth factor concentrations, capacitance, or optical density. In some examples of the presently disclosed system, the sensors provide data at discrete time intervals or continuously.

In some further examples, the system comprises a feedback control component which automatically adjusts one or more physical parameters of the culture media based on the physical attributes of the culture media detected by the sensors.

### EXAMPLE METHOD

### Example Method #1

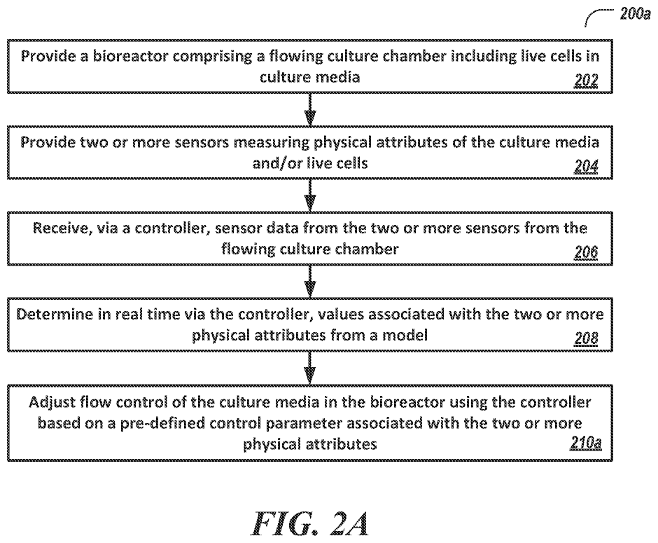

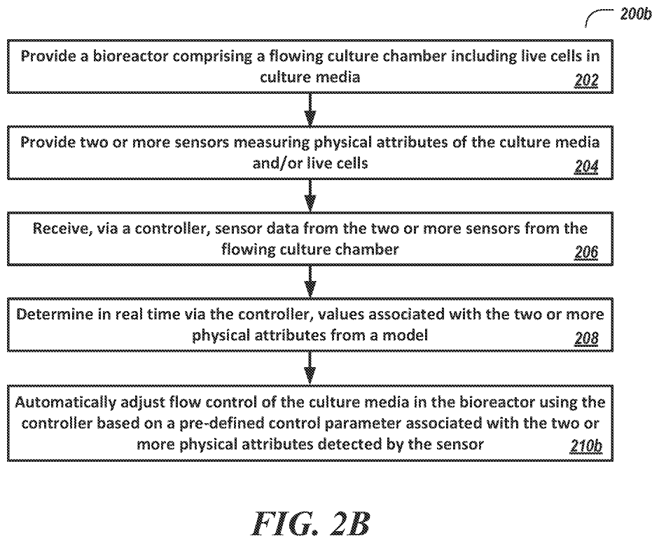

FIG. 2A-2B each show a method (200a, 200b) using model-based control for determining process parameters in a bioreactor in real time (inline).

Referring to FIG. 2A, the method 200a includes providing a bioreactor comprising a flowing culture chamber, including live cells in culture media (202).

Method 200a further includes providing two or more sensors for measuring two or more physical attributes of the culture media and/or the live cells (204);

Method 200a further includes receiving, via a controller, sensor data from the two or more sensors operatively disposed on, or in a flow pathway of, the flowing culture chamber (206);

Method 200a further includes determining, via the controller, in real time, values associated with derived attributes of the culture media or live cells therein using a model employing the sensor data from the two or more sensors (208);

Method 200a further includes adjusting, via the controller, according to a pre-defined control parameter, flow control of the culture media in the bioreactor using the determined values associated with and derived from physical attributes of the culture media or live cells therein.

In some embodiments, the live cells include immune cells, stem cells, stem-derived cells, or red blood cells. For example, in some embodiments, the immune cells comprise chimeric antigen receptor (CAR) T cells, natural killer cells (NKs), T-regulatory cells (T-regs), T cells, or tumor infiltrating lymphocytes (TILs). In some embodiments wherein the live cells include stem cells, the stem cells comprise induced pluripotent stem cells (PSCs), mesenchymal stem cells (MSCs), or MSC- or PSC-derived cells.

According to some embodiments of the present method, the bioreactor comprises a stirred-tank, airlift, hollow-fiber, or rotary cell culture system (RCCS). In some embodiments, the bioreactor is a small-scale or medium-scale bioreactor.

In some embodiments, the two or more physical attribute comprises a critical quality attribute (CQA) and/or a critical process parameter (CPP). For example, in some examples, the two or more different physical attributes comprise dissolved oxygen, pH, glucose level, temperature, conductivity, NO/NOx, volatile organic compounds (VOCs), ozone, chlorine, reduction-oxidation (redox) potential, agitation, inward gas flow, outward gas flow, pressure, vessel weight, metabolite concentrations, cytokine or growth factor concentrations, capacitance, or optical density. In some embodiments of the present method, the sensors provide data at discrete time intervals or continuously.

Method 200a can be performed e.g., using systems described relating to FIG. 1 among others described herein.

### Example Method #2

Referring to FIG. 2B, method 200b performs the operation of steps 202, 204, 206, and 208.

Method 200b further includes automatically adjusting flow control of the culture media in the bioreactor using the controller based on a pre-defined control parameter associated with the two or more physical attributes detected by the sensor (210b).

In some aspects, the method further includes: a feedback control that automatically adjusts one or more physical parameters of the culture media based on the physical attributes of the culture media detected by the sensors. For example, in some aspects, one or more physical parameters are adjusted by the addition of a composition. In some embodiments, the amount or type of adjustment of the physical parameter is variable based on feedback received for that physical parameter.

The designed MSHF bioreactor is inspired by Quantum, where cells attach and proliferate on the inner surface on the fibers while fresh media are continuously fed in from media bag and waste media are removed at the same time. The hollow fibers are made of semi-permeable membranes, allowing small molecules to freely pass through while macromolecules are retained inside of the fibers. Here, the space that passes inside of the fibers is referred to as intracapillary space (IC), while the opposite is referred to as extracapillary space (EC). Media are fed through IC while both IC and EC circulate during the process to provide effective mixing and gas exchange. Pumps and valves are integrated in each loop and can be controlled from the graphic user interface to accomplish automated pre-set tasks. The hollow fiber cartridge from FiberCell systems is almost identical to Quantum in setup but different in dimensions. The fiber membranes from both systems are mainly made of poly-sulfone with the same molecular weight cutoff, and the fibers have the same diameter and wall thickness. However, after assessment, it was found that the fluidic schematic mirrored after the Quantum was not sufficient to mimic the Quantum protocol, and as such, a new set of fluidics was designed for effective scaling.

Sensor technologies are key process analytical technologies that enable the advancement of biomanufacturing by enabling in-line monitoring and feedback control mechanisms. One of the key challenges in cell therapy manufacturing is to understand how the bioreactor environment affects the cell proliferation and quality. pH and dissolved oxygen (DO) have been routinely monitored in the biopharmaceutical production space since these two parameters are long known to affect cell viability. Nutrient and metabolite levels (e.g., glucose and lactate) have also been identified as potential critical process parameters for a lot of cell types as the levels of these molecules can indicate the rate of metabolism, and changing concentrations of these molecules might also shift cell metabolic pathways, altering their functions. Cell therapy-specific parameters, such as endogenous factors, surface markers, etc. have also recently emerged to be potentially tied to final product potency. Thus, monitoring these parameters can deepen the understanding of the process requirement for cell therapy manufacturing and facilitate process analytical technology integration. This disclosure has identified both glucose/lactate sensor (C-CIT) and pH/DO sensor (Scientific Bioprocessing (SBI)) to be integrated in-line with the MSHFB. Both platforms are flow-through.

To create a semi-autonomous bioreactor platform, modeling and feedback controls were integrated to the medium-scale bioreactor platform as proof-of-concept capabilities that advance current operational processes for the biomanufacturing of MSCs. Monitoring of metabolites such as glucose and lactate in the cell culture media are current approaches to evaluate the expansion process and growth of the cells. By manually sampling or use of in-line sensors, glucose, and lactate levels can be input into developed models that inform control mechanisms for directly altering the feed rate (i.e., media perfusion) for maintaining metabolite levels throughout the expansion.

For those metabolites which cannot be monitored in-line, an automated sampling platform was designed to automate the manual processes required to collect samples of media during an expansion. By automating the sampling process, reductions in risks (e.g., contamination), labor, and perturbations to the bioreactor improve the procedural and product results of biomanufacturing while enhancing the precision and frequency of sampling on a scheduled program. Among other things, the present disclosure discusses the hardware and software for the platform, which is capable of extracting samples from a septum port placed on the EC waste line and storing them into vials for offline analysis. The platform is controlled through a graphical interface and has the capability to take multiple samples at fixed interval throughout the duration of the expansion.

### EXAMPLES

### Example Embodiments

### Example #1: Automation for the Improved Efficacy, Yield, and Reproducibility of the Manufacturing of Human Umbilical Cord Tissue Mesenchymal Stromal Cells for Clinical Therapeutic Use

A small-scale hollow-fiber bioreactors is disclosed that expands MSCs by reproducing scaled process parameters used in the Terumo Quantum system. Initial data of a limited sample size suggests alterations to MSCs when expanded in hollow-fiber bioreactors. Further analysis with increased sample sizes will determine what may be correlated to reduced IDO activity. In the study, glucose and lactate sensors are integrated onto the FiberCell bioreactor for implementing feedback control. Real-time feedback control can be implemented to eliminate spikes in glucose and lactate in the culture media. Cell density sensors can be employed to produce different resistance signals with respect to different locations within the cartridge.

The system can be employed to expand potent MSCs. The system can be used on cells to produce therapeutic levels of a secreted factor(s) that may be application dependent. The system can lower cost and risk through scalable and reproducible culture.

Background Human umbilical cord tissue mesenchymal stromal cells (hCT-MSCs) have demonstrated promise in several clinical trials for patients with unmet medical needs. The high interest and demand for hCT-MSCs underscore an increased need to transition their manufacturing from small-scale static cultures to larger-scale bioreactor-driven processes. To enable scalable translation while ensuring consistent, high-quality products, rigorous identification of critical quality attributes (CQAs) and critical process parameters (CPPs) that not only ensures target release characteristics but also predicts cell potency and safety while reducing the cost of goods is required. Bioreactor-based expansion systems are able to monitor CQAs and modulate CPPs in real time with automation implemented, enabling consistent product quality and lowering cost at an industrial scale.

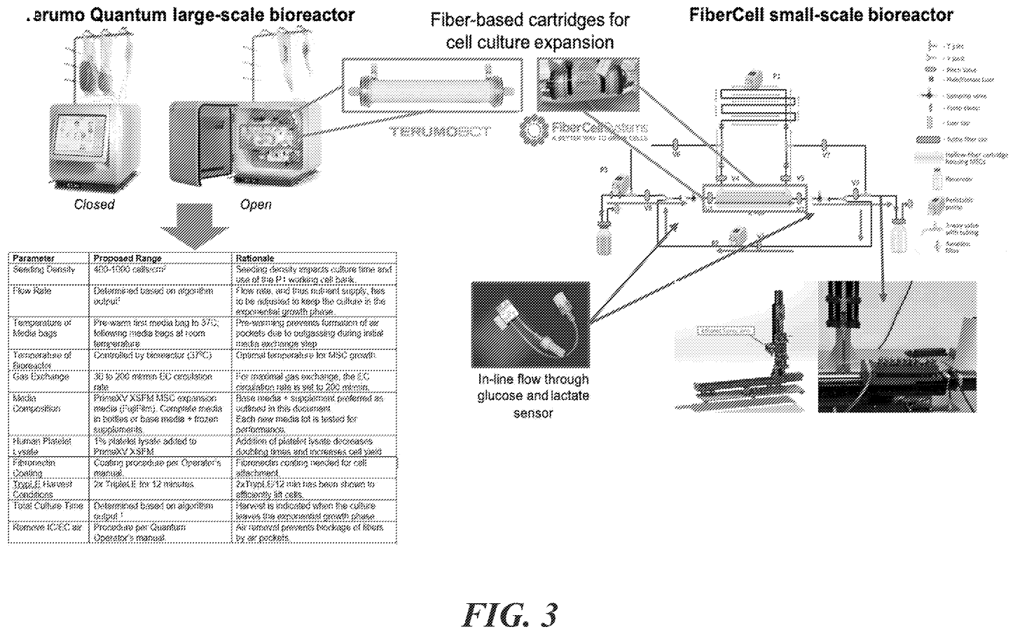

A small scale hollow-fiber bioreactors is disclosed that expands MSCs by reproducing scaled process parameters used in the Terumo Quantum system. FIG. 3 shows an fiberCell bioreactor to which a model can be generated.

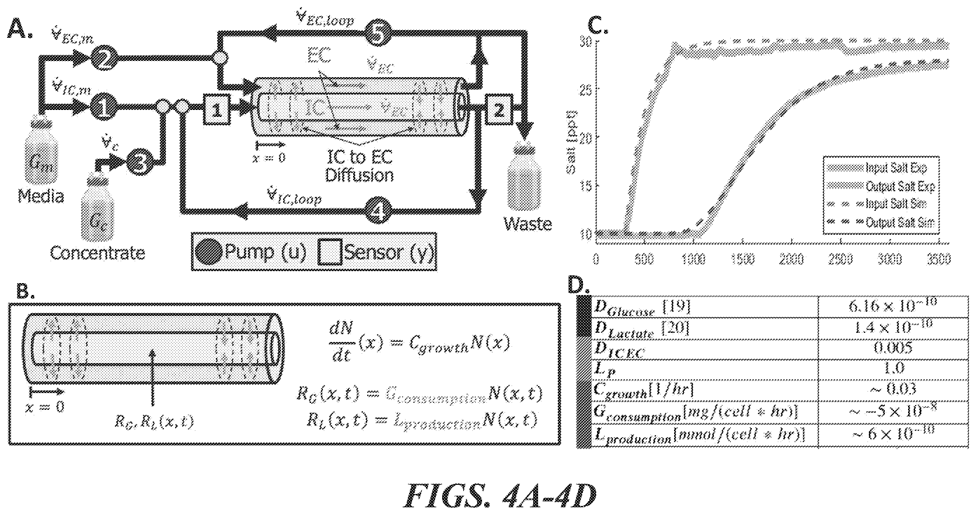

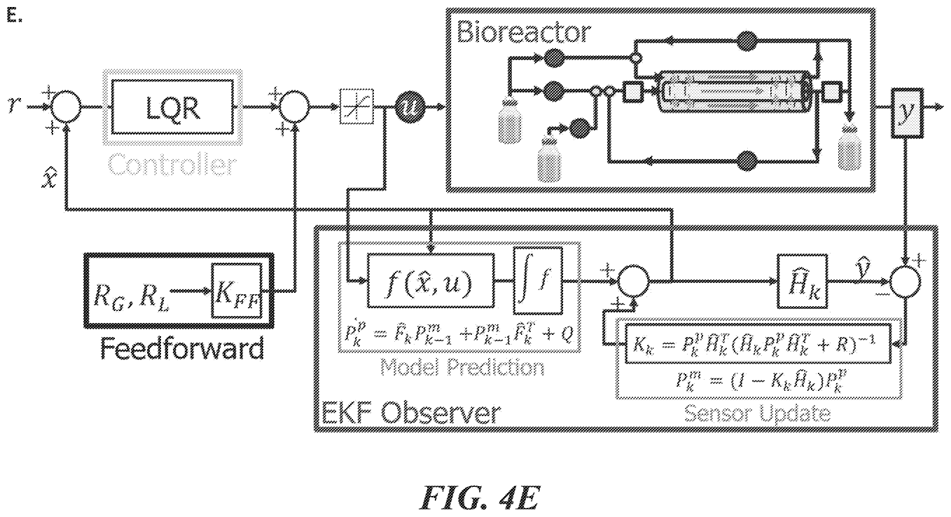

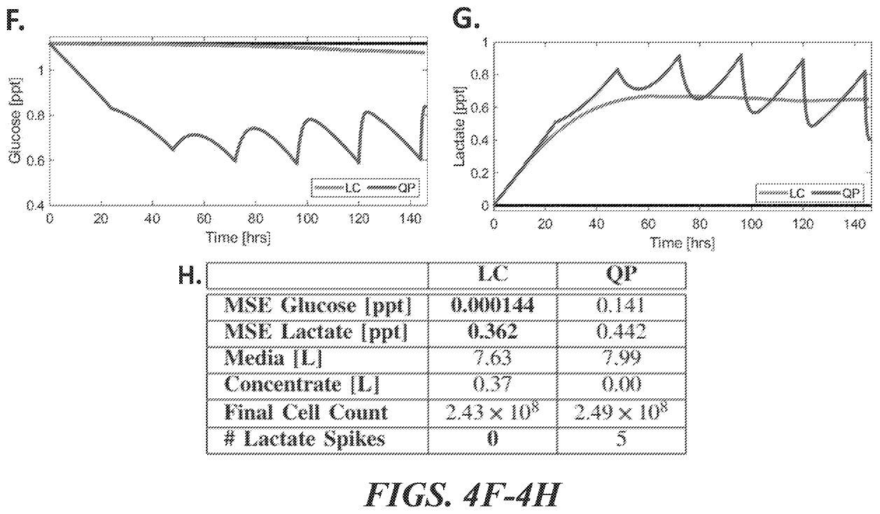

Modeling and Controls. FIGS. 4A-4H describe the modeling and control of the fiberCell bioreactor of FIG. 3. In the model and system, glucose and lactate sensors are integrated onto the FiberCell bioreactor for implementing feedback control. Real-time feedback control can be implemented to eliminate spikes in glucose and lactate in the culture media.

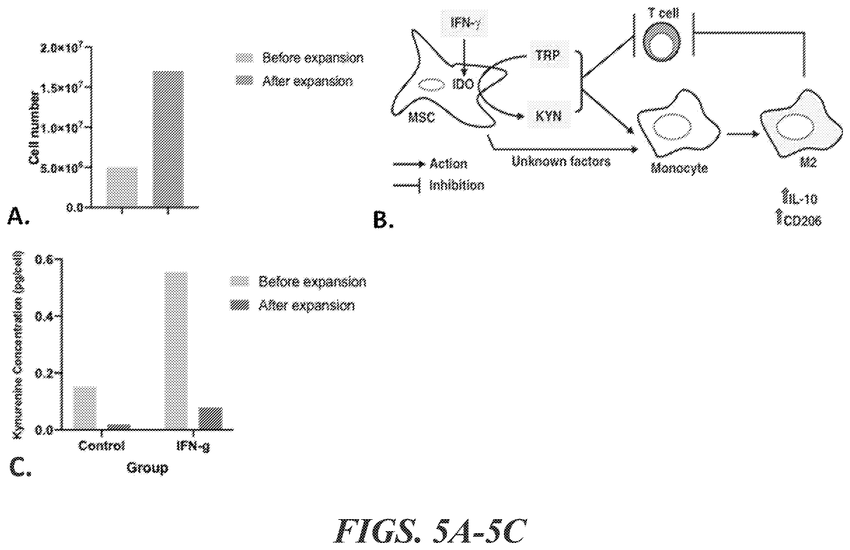

MSC Expansion with FiberCell BioReactor. FIGS. 5A-5C show MSC expansion was carried out on a hollow-fiber bioreactor model. Initial data of a limited sample size suggests alterations to MSCs when expanded in hollow-fiber bioreactors. Further analysis with increased samples sizes may determine correlations to reduced IDO activity.

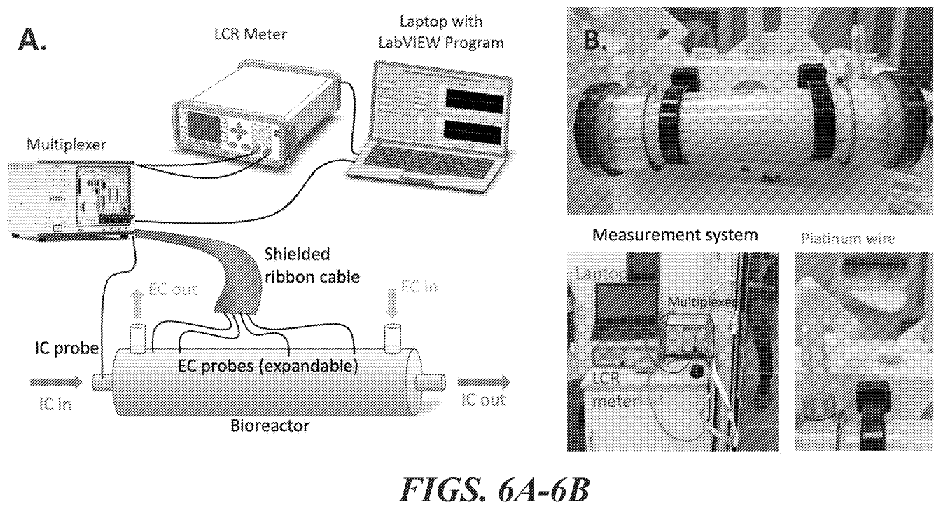

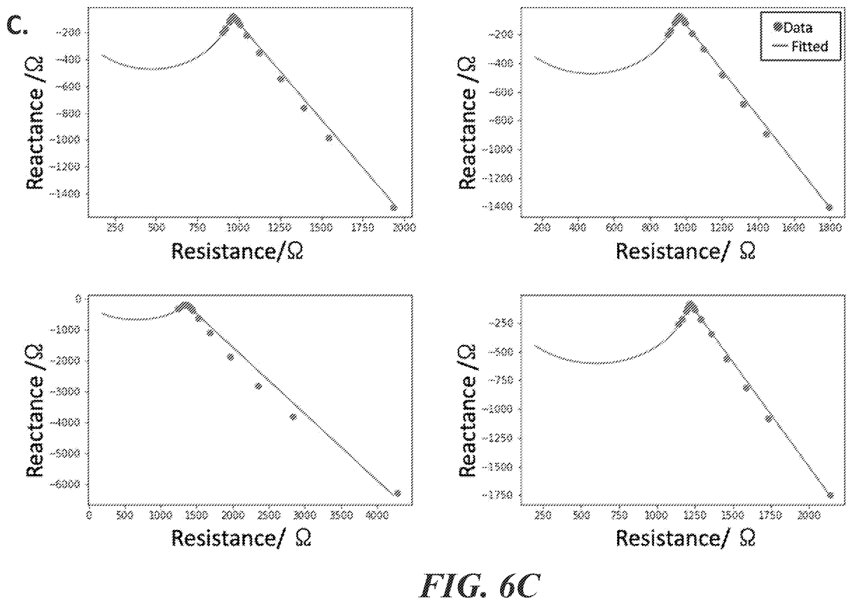

Cell Density Sensor. Cell density sensors can be employed to produce different resistance signals with respect to different locations within the cartridge. FIG. 6 shows a schematic for cell density sensing system.

### Example #2: Automation for the Improved Efficiency, Yield, and Reproducibility of the Manufacturing of hCT-MSC

A Bioreactor system is shown that can be implemented for small to medium-scale bioreactor models for the expansion of MSCs. The bioreactor system comprises a vertical wheel bioreactor that can achieve higher and more consistent product yield after optimization compared to an unoptimized protocol. The FiberCell has the potential to be the scaled-down version of the Quantum bioreactor. The design was optimized using the design of experiment approach. Flow-through glucose and lactate sensors can be integrated into the FiberCell system at both the inlet and outlet. The system includes a customized cell density sensor to produce a sensible response with cells in a bioreactor cartridge.

A system can employ wireless sensor technology to continuously monitor glucose/lactate levels and cell growth characteristics.

The system includes an automation function that can create automated and responsive unit operations for maintaining culture efficiency, increasing culture reproducibility, and reducing labor requirements. The automated controller is able to eliminate metabolite spikes in the media profile, keeping glucose levels high and lactate levels low, potentially creating high-quality MSC products.

Background Human umbilical cord tissue mesenchymal stromal cells (hCT-MSCs) have demonstrated promise in several clinical trials for patients with unmet medical needs. The high interest and demand for hCT-MSCs underscore an increased need to transition their manufacturing from small-scale static cultures to larger-scale bioreactor-driven processes. To enable scalable translation while ensuring consistent, high-quality products, rigorous identification of critical quality attributes (CQAs) and critical process parameters (CPPs) that not only ensures target release characteristics but also predicts cell potency and safety while reducing the cost of goods is required. Bioreactor-based expansion systems are able to monitor CQAs and modulate CPPs in real time with automation implemented, enabling consistent product quality and lowering cost at an industrial scale.

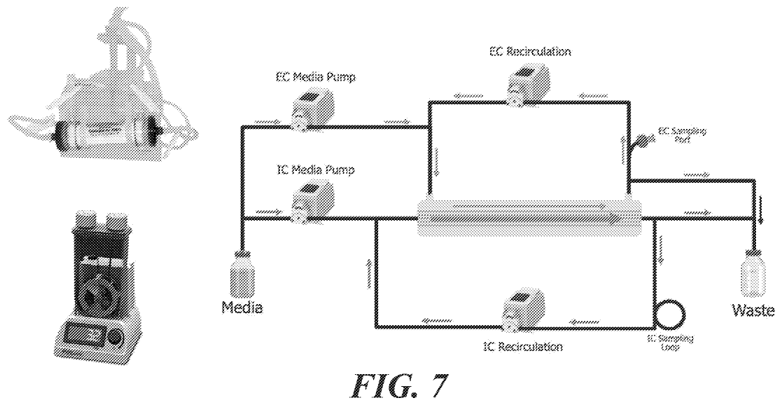

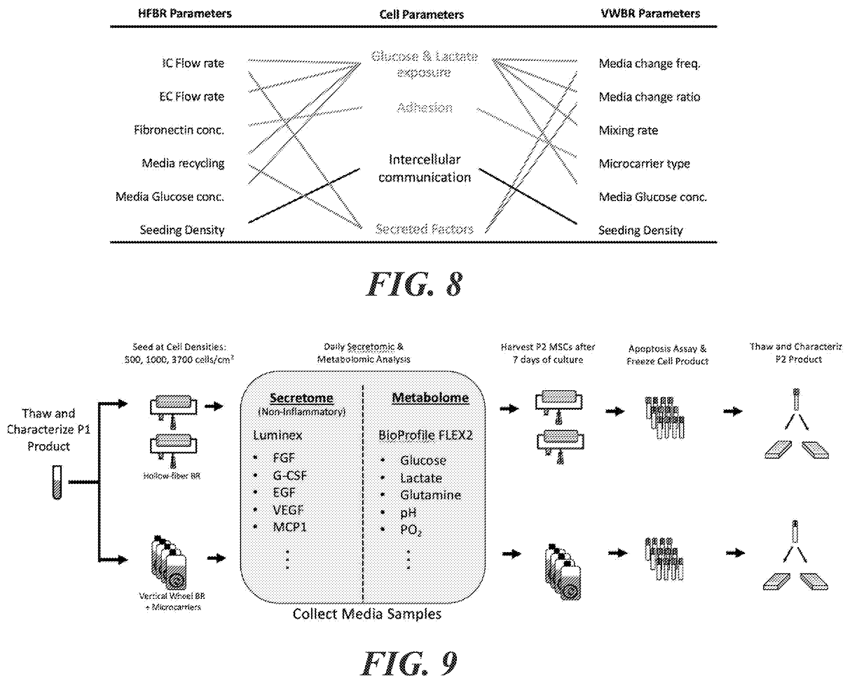

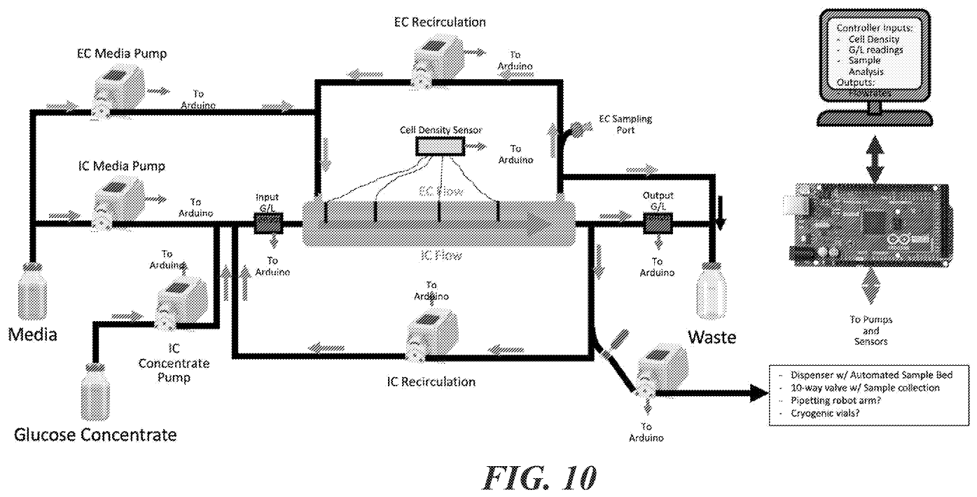

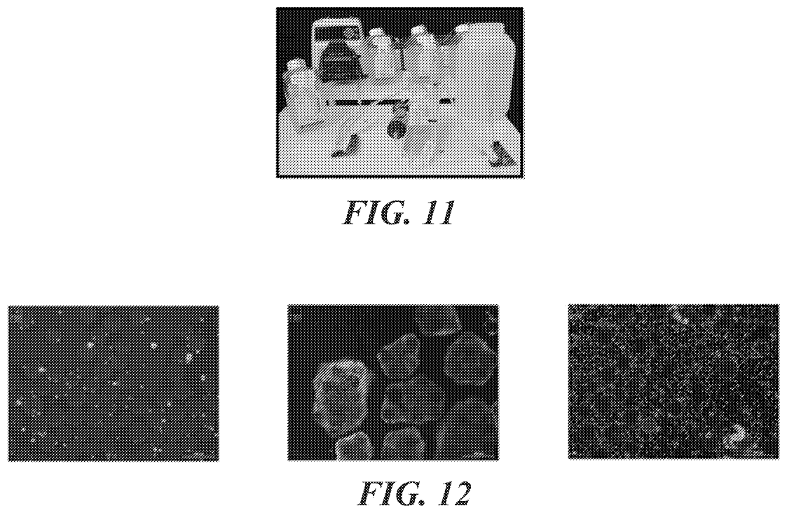

FIG. 7 shows an example bioreactor design for the expansion of MSCs. The models include the hollow fiber model and the vertical wheel model. FIG. 8 shows an optimization approach taken employing fractional factorial design and response surface methodology. FIG. 9 shows an example processing that can be employed by the system. FIG. 10 shows an automation function that can create automated and responsive unit operations for maintaining culture efficiency, increasing culture reproducibility, and reducing labor requirements. The automated controller is able to eliminate metabolite spikes in the media profile, keeping glucose levels high and lactate levels low, potentially creating high-quality MSC products. FIG. 11 shows an experimental setup of surrogate bioreactor systems.

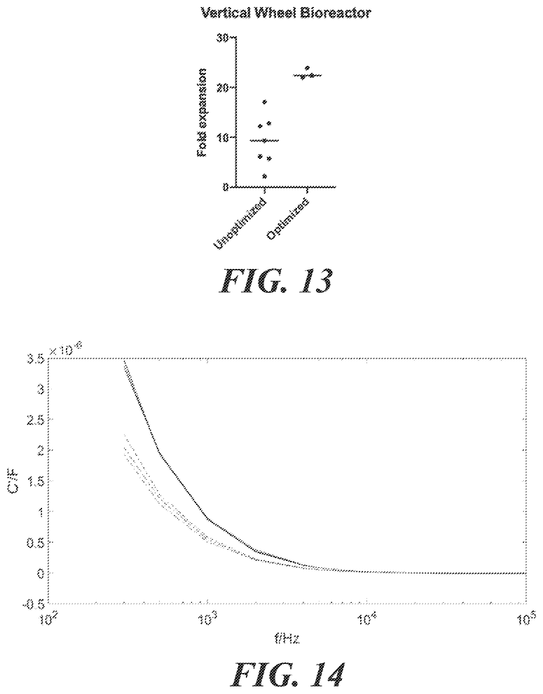

Experimental Results. FIG. 12 shows that the vertical wheel bioreactor can be optimized to be a suitable culturing method for CT-MSCs in a serum-free condition. FIG. 13 shows the results of the vertical wheel bioreactor between optimized and unoptimized configurations. FIG. 14 shows that a customized cell density sensor can pick up responses with cells attaching on fibers.

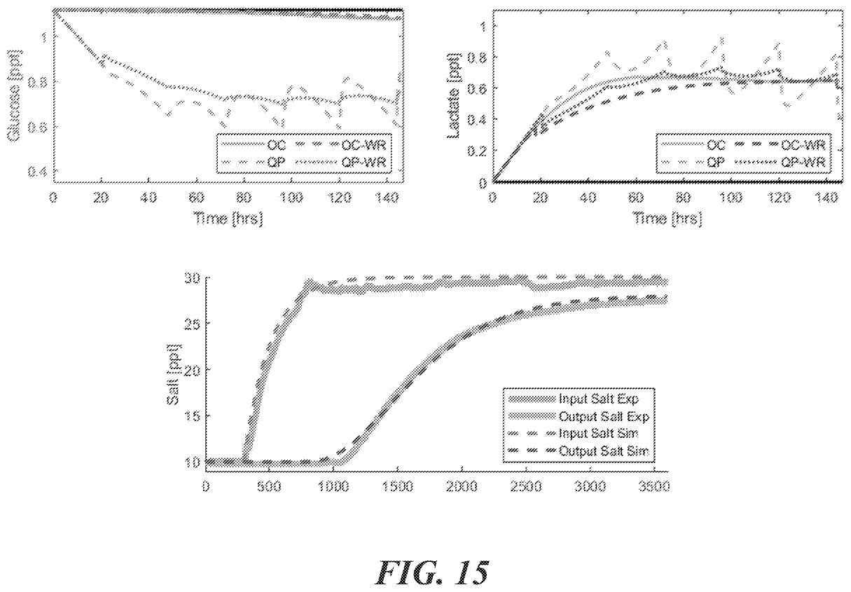

From the experiment, it can be observed that the automated controller is able to eliminate metabolite spikes in the media profile, keeping glucose levels high and lactate levels low, potentially creating high-quality MSC products. In FIG. 15, it can be observed that the automation approach can reduce process disturbances, and experimental results match simulation results.

### Example #3: Advanced Manufacturing Process Design for Mesenchymal Stromal Cell Therapy

FiberCell can be used as a scale-down model for MSC expansion with high cell recovery without reducing cell potency. Real-time feedback control can be integrated into FiberCell and can eliminate spikes in culture media during expansion and improve yield. Cell density sensors can detect the existence of cells within the hollow fibers through analysis.

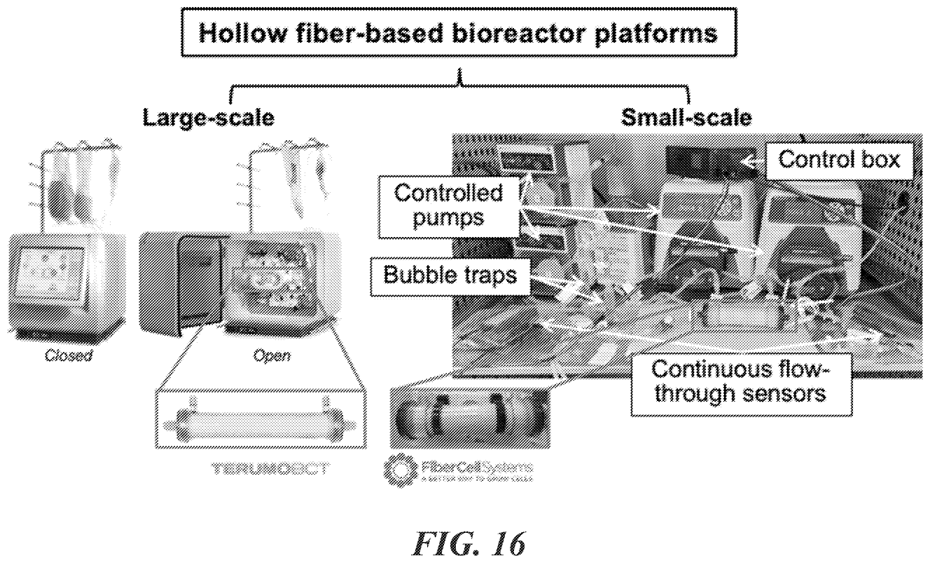

FIG. 16 shows a hollow-fiber bioreactor that includes a novel dosing solution, inlet and recirculation flowrates, and input/output combined glucose and lactate sensors.

Background. Human umbilical cord tissue mesenchymal stromal cells (hCT-MSCs) have demonstrated promise in several clinical trials for patients to treat various diseases. The high interest and demand for hCT-MSCs underscore an increased need to transition their manufacturing from small-scale static cultures to larger-scale bioreactor-driven processes. In this study, a “smart” feedback-controlled hollow fiber-based bioreactor was designed and tested for maintaining nutrient and waste levels for hCT-MSC expansions. The bioreactor platform is a semi-autonomous system complete with in-line sensors, modeling, data-driven controllers, and an automated sampling platform. The small-scale system reduced costs, labor, time, and perturbations and improved yields of MSC products using a hollow fiber cartridge that closely models the basic design of the large-scale Quantum® Cell Expansion System.

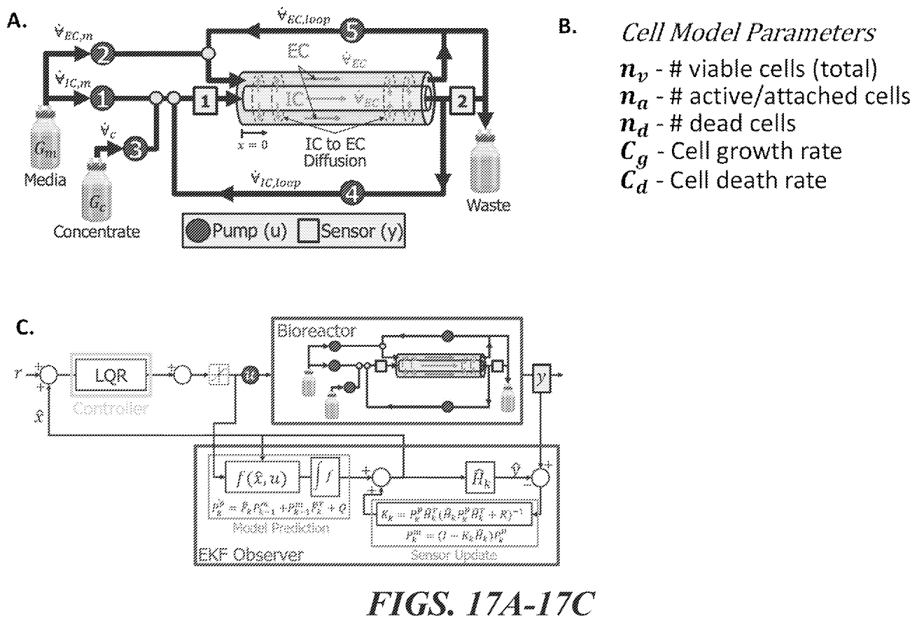

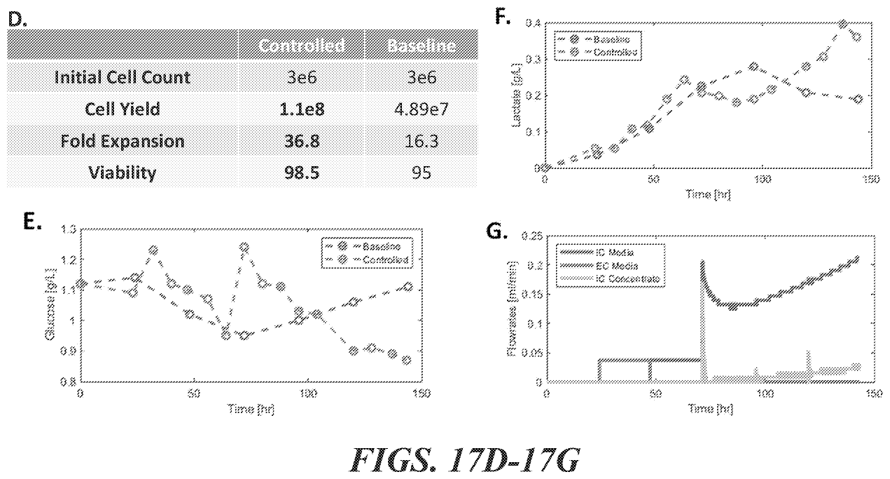

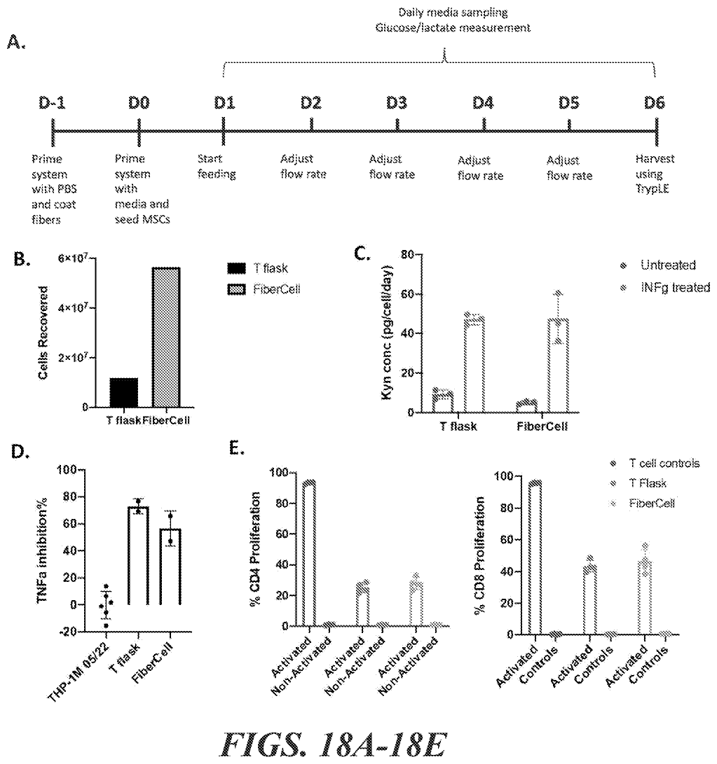

Modeling and Control. FIG. 17A-17G shows a model for hollow-fiber bioreactors of FIG. 16 and experimental results. FIG. 18A-18E shows a method of operation for the MSC expansion process.

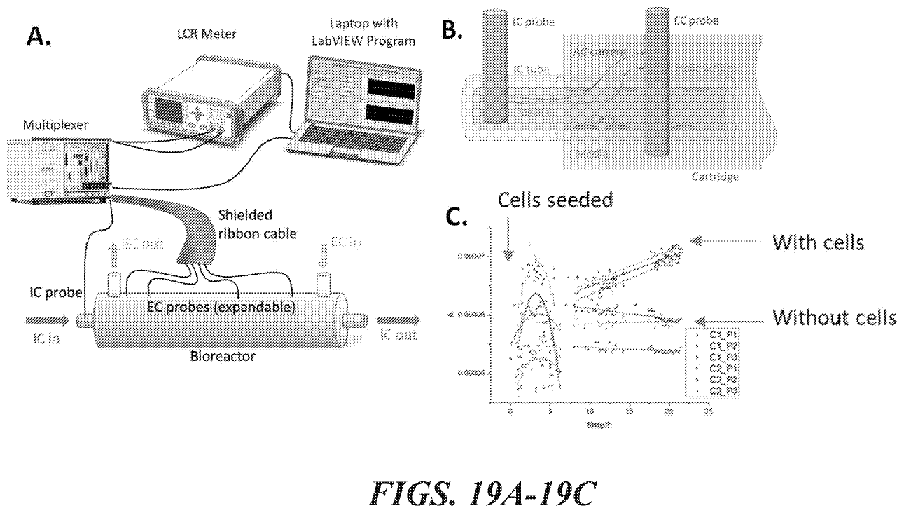

Cell Density Sensor. Cell density sensors can detect the existence of cells within the hollow fibers through analysis. FIGS. 19A-19C shows a schematic for the cell density sensing system and bioreactor integration.

### Example #4: Digital Twin Design for hMSC Expansion in Hollow-Fiber Bioreactors

Background Adult human Mesenchymal Stromal Cells (hMSCs) offer promising therapeutic effects for regenerative medicine. Pre-clinical studies have demonstrated immunomodulatory results from hMSC treatment for various indications [1]-[3]. Automated cell-expansion platforms and consistent quality cell products are necessary to scale up production to further study these clinical effects [4], [5].

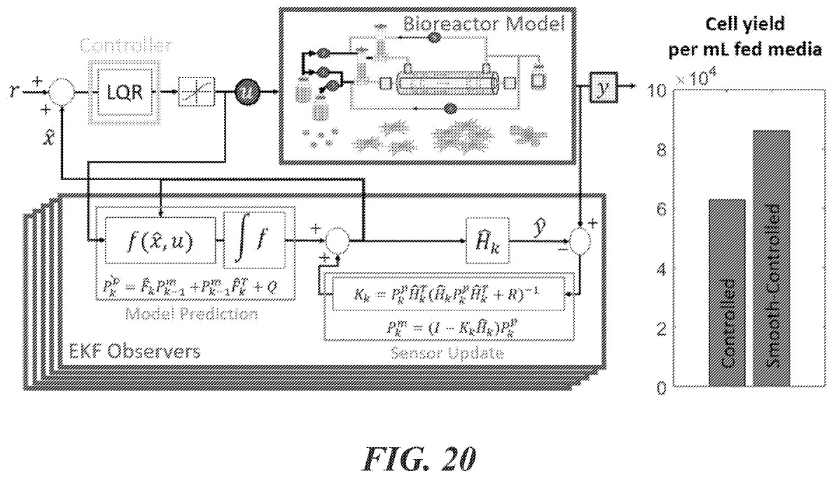

Hollow-fiber bioreactors, such as the Quantum (Terumo BCT), are approved for cell manufacturing for clinical use to enable the large-scale expansion of hMSCs [6]. Bioreactors attempt to regulate the many relevant processes and environmental parameters in the cell expansion process. Currently, these quantities are regulated with intermittent intervention from skilled operators to measure nutrient values and update flow rates [7]. This method results in slow feedback rates, limited knowledge of full system dynamics, and increased variability. Automated control and modeling techniques have been proposed for MSC growth [8], [9] but have so far been limited to small-scale, static culture vessels. [10]. Described in this example are additional adaptations to the biomanufacturing processes discussed in the earlier examples, including 1) updating the control-centric model with updated fluid dynamics parameters and time-varying cell dynamics, 2) designing an estimation model for cell count and cell growth rate parameters, 3) formulating a multiple observer model that accounts for new sensing frequency limitations and estimates both unmeasurable states and model parameters, 4) upgrading the state feedback controller to improve glucose and lactate regulation, and 5) show that the updated estimation and control architecture improves cell yield per mL fed media over another control architecture in cell expansion (FIG. 20).

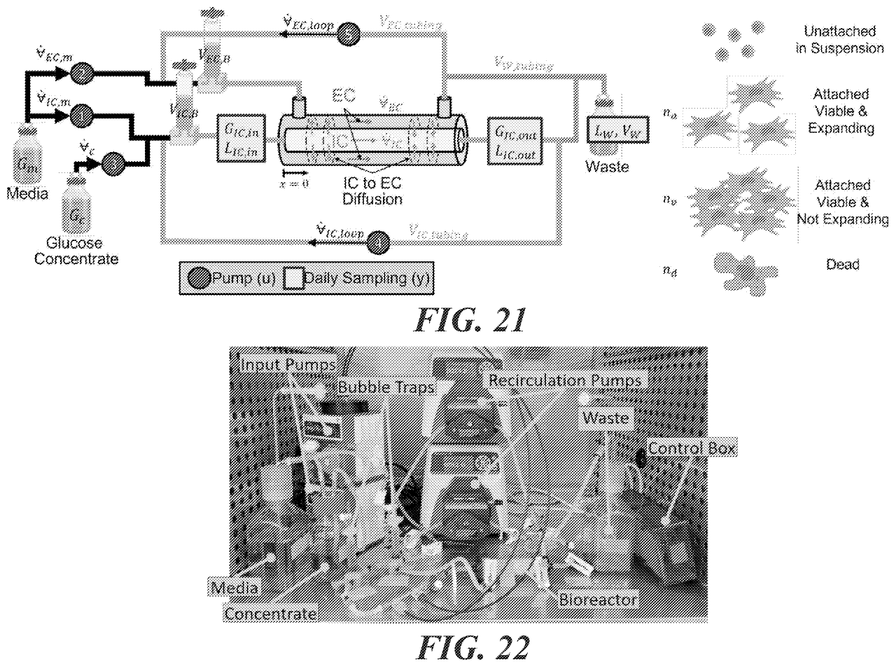

One aspect of the present disclosure is to regulate the concentrations of glucose and lactate within the bioreactor. The bioreactor setup also remains largely the same as in earlier examples, with the medium-scale hollow-fiber bioreactor's intra-capillary (IC) space as a bundle of semi-permeable fiber membrane tubes in which hMSCs are seeded and an extra-capillary space (EC) which allows for parallel media flow and nutrient exchange. The same media and glucose concentrate solutions were used to perfuse across the cells and replenish nutrients/remove waste during the expansion process. Peristaltic pumps were used for all five actuators shown in FIG. 21.

The exemplary bioreactor system introduced new fluid dynamics in the form of modified IC and EC flow loops with reduced volumes, which reduced the loop flow rates. It also introduced 5 mL dead volumes in the IC and EC loops, which trap bubbles out of the bioreactor and reduce input nutrient concentration spikes. In-line continuous glucose and lactate sensors were replaced with a daily at-line sampling of glucose and lactate at the inlet and outlet due to inconsistency of sensor calibration and long-term inaccuracy of data. Additional daily measurements of waste media lactate and volume were also taken for improved observer performance. This exemplary bioreactor setup is shown in FIG. 22 and is used for experimental cell expansion results.

This study compared the performance of an automated control architecture (smooth-controlled) with both a baseline manual expansion protocol (baseline) and a prior automated control architecture (controlled). All three protocols start with coating and seeding the bioreactor. After this step, the baseline protocol introduces a minimal level of perfusion for the first 2 days of expansion, first through EC, then through IC, and doubles flow rates every day after that until the end of the expansion on day 6. The prior automated control design followed the same baseline protocol until day 3 after which the controller regulates input media and concentrate flowrates. In contrast, the proposed controller began perfusion on day 1 itself with fixed setpoints at the day 1 glucose and lactate measurement levels. All protocols were also limited to 1 L of media for perfusion. The protocols were compared with the following metrics: Glucose and lactate setpoint mean squared error (SMSE), Glucose and Lactate observer mean-squared error (OMSE), media usage, # of lactate spikes, and final cell yield.

In order to implement automated feedback process control, the experiment first designed a digital model for the physical plant (a medium-scale hollow-fiber bioreactor) that accurately fits the inputs (media flowrates), in-process measurements (glucose and lactate), and cell number at the end of the expansion. The study then applied optimal estimation and control algorithms to interface with the physical plant and control for specific process parameters. This procedure has been documented for other bio-manufacturing processes under a digital-twin modeling framework [11]. This modeling task is split up into a fluid dynamics model, a cell dynamics model, a cell expansion estimation model, an observer model for optimal estimation, and a controller.

Fluid Dynamics Model. The fluid dynamics model of the bioreactor describes how concentrations of glucose and lactate vary due to bioreactor design/geometry and media flow rates. The IC and EC states for the hollow-fiber bioreactor were designed and validated in a prior work, and the dynamics describing the convection-diffusion model for glucose and lactate are shown below:

$$\frac{\partial G}{\partial t} = {D}_{G} \frac{{\partial }^{2} G}{\partial {x}^{2}} + \frac{A}{} \frac{\partial G}{\partial x} + {K}_{ICEC} \frac{{\partial }^{2} {G}_{ICEC}}{\partial {r}^{2}} + {G}_{c} {n}_{a} \qquad ( 1 )$$

$$\frac{\partial L}{\partial t} = {D}_{L} \frac{{\partial }^{2} L}{\partial {x}^{2}} + \frac{A}{} \frac{\partial L}{\partial x} + {K}_{ICEC} \frac{{\partial }^{2} {L}_{ICEC}}{\partial {r}^{2}} + {L}_{p} {n}_{a} \qquad ( 2 )$$

The parameter determination, discretization, and model validation are described in more detail in [10]. The exemplary bioreactor setup in FIG. 21 introduced new tubing volumes, VIC,tubing=0.01 L, VEC,tubing=0.025 L, and VW,tubing=0.01 L. For the flow rates studied in this experiment, the volumes were sufficiently mixed with minimal gradients when simulated. Therefore, these volumes were modeled as continuously mixed, so like with the sensor and tubing states in [10], a mixed-tank linear model was used for these additional volumes.

The new dead volumes, VIC,B and VEC,B, were not necessarily constant during the expansion as media evaporates and bubbles get trapped in the dead volume. In application, however, the dead volume states were modeled as constant (at 0.004 L) due to the back-pressure from the hollow-fiber bioreactor filling up the volumes, and could, therefore also use the mixed-tank linear model approach. The evaporated media and bubble volume then resulted in reduced waste bottle volume. Ordinarily, this evaporation rate would need to be modeled. However, the waste bottle measurement serves the specific model purpose of determining the cumulative lactate quantity (mg) in the waste bottle, which would not be affected by evaporation and bubbles. Therefore, the newly added waste bottle states, VW and LW, were also modeled using the mixed-tank linear model as follows:

$$\frac{{dV}_{W}}{dt} = {IC , m}_{} + {EC , m}_{} + {c}_{} \qquad ( 3 )$$

$$\frac{{dL}_{W}}{dt} = \frac{{L}_{W , tubing}}{{V}_{W}} ( {IC , m}_{} + {EC , m}_{} + {c}_{} ) \qquad ( 4 )$$

Cell Dynamics Model. In [10], glucose consumption and lactate production terms were driven by a simple exponential growth function for cell number with constant cell growth rate, glucose consumption rate, and lactate production rate. The experiment advanced this model to account for varying growth parameters and growth phases.

The main parameters/states of interest for this improved cell model (FIG. 21) are as follows:

$$θ = [ {n}_{v} ; {n}_{a} ; {n}_{d} ; {C}_{g} ; {C}_{d} ] \qquad ( 5 )$$

with total viable cells (nᵥ), attached/actively growing cells (nₐ), dead cells (n_{d}), cell growth rate (C_{g}), and cell death rate (C_{d}). For cell growth dynamics, the study split this model into two distinct phases, growth during attachment and growth after attachment.

The attachment phase occurred right after cells were seeded into the bioreactor, at which point all elements of θ were 0. Static hMSC culture indicated that the attachment occurred at a negative exponential rate [12], where 60-90% of the attachment occurred in the first 5 hours and 100% attachment occurred at 24 hrs. This was captured by the following differential equation:

$$\frac{{dn}_{a}}{dt} = \frac{{n}_{seeded}}{14400} {e}^{- t / 14400} \qquad ( 6 )$$

where n_{seeded} was the number of suspended cells introduced into the bioreactor. Once the cells attach, they begin proliferating, consuming glucose and producing lactate. This was modeled proportionally to the attachment negative exponential with the final cell growth rate being the initialized growth rate value from a prior baseline expansion (C_{g}⁰):

$$\frac{{dC}_{g}}{dt} = \frac{{C}_{g}^{0}}{14400} {e}^{- t / 14400} \qquad ( 7 )$$

The attachment phase ended when na≥0.99nseeded.

After the attachment phase, the cell growth rate starts to drive the majority of the glucose decrease and lactate increase in the bioreactor. To compute nₐ in this phase, the study first accounted for cell confluency (C_{c}), computed as the fraction of attached MSC surface area (S_{C}) to available fiber surface area (S_{f}), which can inhibit expansion at high cell densities. This assumes even distribution of cells across the fibers.

$${C}_{c} = \frac{{n}_{v} {S}_{C}}{{S}_{f}} \qquad ( 8 )$$

Cells are mobile during growth and will aim to expand into empty space, so with an even initial seeding, the confluency should be relatively uniform across fibers. MSCs are typically harvested at 70-80% confluency [13]. With this notion, the study computed nₐ or the number of attached cells that could double given the available surface area as follows:

$${n}_{a} = \{ \begin{matrix}{n}_{v} & if 0 \leq {C}_{c} < 0.5 \\ ( 0.8 - {C}_{c} ) {n}_{v} & if 0.5 \leq {C}_{c} < 0.8 \\ & else harvest cells\end{matrix} \qquad ( 9 )$$

FIG. 21 highlights this confluency effect which differentiates nₐ from nᵥ. In this growth phase after attachment, the following differential equation tracks the total number of viable cells (nᵥ),

$$\frac{{dn}_{v}}{dt} = {n}_{a} ( {C}_{g} - {C}_{d} ) \qquad ( 10 )$$

High confluency, pressure, and shear stress can influence cell death [14]; however the current experimental protocol aimed for 80% confluency at harvest to prevent cell death due to overcrowding while minimizing input flow rates to reduce the effects of changing pressure and shear stress. Therefore cell death was assumed to be negligible for the following simulations and expansions.

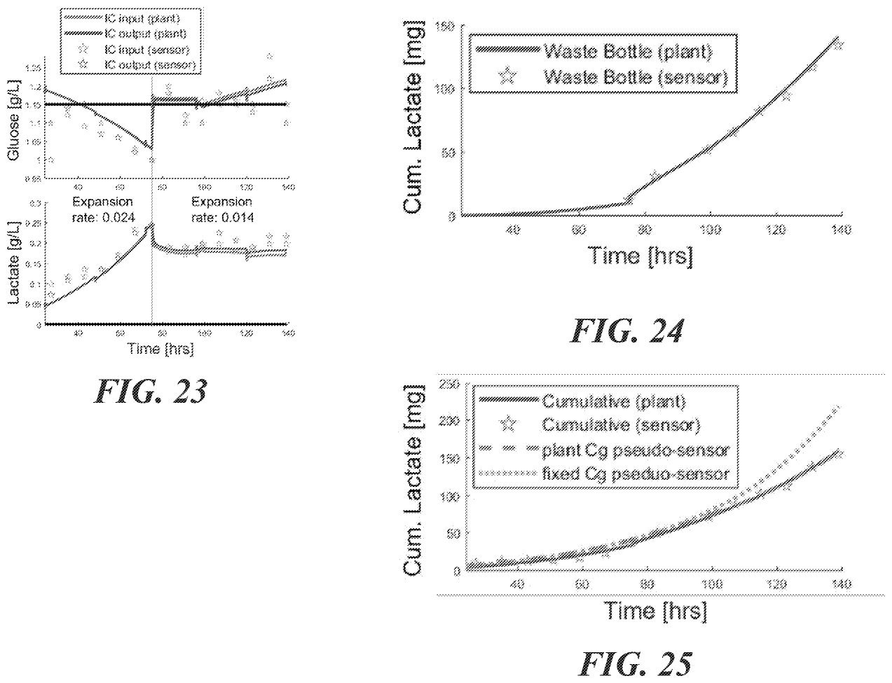

Fluid and Cell Model Validation. The exemplary fluid dynamic and cell dynamic models were validated with a preliminary controlled cell expansion. The cell model parameters were initialized to the rates observed in prior baseline expansions. Measurements of the input and output glucose and lactate concentrations were taken and compared to the fitted model (FIG. 23). Additionally, with this best-fit parameter model, the waste cumulative lactate (defined as V_{w}L_{w}) was simulated and compared with the measured lactate concentrations and volumes in the waste bottle (FIG. 24) with a best-fit MSE of 0.0114 mg for waste cumulative lactate. From FIG. 23 it is clear that the best-fit model for C_{g} denoted a transient cell growth rate, which indicated either a future dynamic model for C_{g} or an effective estimator. With the exception of the C_{g} parameter all other fluid and cell model parameters were validated.

Cell Expansion Estimation Model. As discussed in the previous section, incorrect cell model assumptions, especially with infrequent samples, can result in reduced predictive accuracy. Therefore, the cell dynamics model was further extended to explore cell growth rate and cell number estimation based on in-process metabolite measurements. Work has been conducted with perfusion-based bioreactors to correlate lactate mass flux to measured cell number [9]. However, this fitted correlation model was limited because it relied on accurate and frequent lactate measurements without allowing for cell growth rate estimation for forward simulation. The described dynamic model addressed both issues.

Since the bioreactor was a closed system, all lactate present in the reactor, tubing, and waste was assumed to have been produced by cells in the bioreactor. With the addition of the waste bottle sensed states it is possible to estimate the cumulative lactate at sample-time k:

$$\begin{matrix}{L}_{cum}^{k} = {L}_{ω} {V}_{ω} + {L}_{IC , out} {V}_{W , tubing} + {L}_{IC , out} {V}_{W , tubing} + \frac{{L}_{IC , in} + {L}_{IC , out}}{2} \\ ( {V}_{IC , tubing} + {V}_{EC , tubing} + {V}_{IC , B} + {V}_{EC , B} )\end{matrix} \qquad ( 11 )$$

This computation cannot take advantage of the estimated states since those are based on potentially incorrect model assumptions. In order to compute cell growth rate, an equation was needed to relate cumulative lactate, cell growth rate, and viable cell number shown below:

$${L}_{cum}^{k} = α \frac{{L}_{p} {n}_{υ}^{k}}{{C}_{g}^{k - 1}} ( {e}^{{C}_{g}^{k - 1} ( {t}^{k} - {t}^{k - 1} )} - 1 ) + {L}_{cum}^{k - 1} \qquad ( 12 )$$

$${n}_{υ}^{k} = {n}_{υ}^{k - 1} {e}^{{C}_{g}^{k - 1} ( {t}^{k} - {t}^{k - 1} )} \qquad ( 13 )$$

where C_{g} and nᵥ are initialized to the baseline fitted cell growth rate and the initial seeded cell number, respectively. Equation 12 can be considered a pseudo-sensor for C_{g}^{k-1} and can be solved by using Newton-Raphson root-finding methods. Equation 13 is also a pseudo-sensor for n^{k}ᵥ as a function of the solution of Equation 12 for C_{g}^{k-1}. The same bioreactor expansion shown in FIG. 23 was used to validate the cumulative lactate pseudo-sensor. FIG. 25 shows Equation 12 plotted with the “true” cell growth parameters as the plant C_{g} pseudo-sensor, while the observer C_{g} pseudosensor equation assumes that the cell growth rate stayed constant at the initialized value for the entire expansion. The plant C_{g} pseudo-sensor also matched well with the computed cumulative data (MSE of 0.0112 mg), although there was some variance attributed to the lactate sensor and volume measurement noise.

Observer Model. The above fluid dynamics, cell dynamics, and cell estimation models enumerate many states that cannot be directly measured. Concentrations and cell counts within the IC fibers or the EC space cannot be accessed due to sterility concerns. Meanwhile, input/output states can be accessed for measurement, but continuous flow-through sensors were inaccurate and inconsistent, while manual measurements were limited to the daily sampling of 1-2 mL to prevent unnecessary cell disturbance. These manual samples are also affected by temperature, which can result in noisy/inaccurate measurements. Therefore it is important to design a state/parameter estimation model that can leverage limited sensing of the bioreactor to best estimate the value of the remaining model states.

In the previous sections, model equations were defined for all of the glucose, lactate, and cell states (X), cell model parameters (θ) (shown in equation (5)), and input flowrates (u). In [10], a noise-optimal state estimate problem was already posited for the glucose and lactate concentration states X. By adding the discretized parameters θ as additional states for the observer architecture to track, the study used the same extended Kalman filter (EKF) design to compute noise optimal estimated parameters in real time.

However, these added parameters θ nearly double the number of states, which results in an unstable EKF model due to the ill-conditioned inverse caused by the variance matrix (P) described in [10]. In order to combat this issue, a multiple partial EKF model was designed, which grouped together only the relevant states for estimation for each observer. Four independent observers were designed to estimate the following sets of states: 1) glucose IC, EC, and tubing, 2) lactate IC, EC, tubing, and waste, 3) cell growth rate (C_{g}), and 4) the number of viable cells (nᵥ).

The glucose and lactate IC and EC observers both used a diagonal R matrix with sensor noise of 0.1 g/L and a diagonal Q matrix with model noise of 0.001 g/L for the best tracking response, very similar to prior work. The waste volume is currently measured visually with a precision of 25 mL, which is used as the diagonal entry in the lactate R matrix. The cell growth rate observer's R-value should be calculated from the measured lactate states; however, this value in the pseudo-sensor (Equation 12) is very unstable for small changes in concentration, so for practicality, a value of 0.01/hr was used. The corresponding model Q value was set to 0.005/hr and the initialized variance was set to 1. The viable cell number observer R-value used the propagated C_{g} R-value through the nᵥ pseudo-sensor (Equation 13 and the Q value was set to 10⁵

Feedback Controller. In this work, a Linear Quadratic Regulator (LWR) controller was used in a manner similar to a previous work [10] to automatically update input bioreactor flow rates at 1 Hz. One aspect improvement works by linearizing and reoptimizing the feedback gains once per day after the in-line sensor measurements are taken. This is advantageous because the present model has continuously changing parameters. The linearization and gains computation was carried out on an Intel NUC 8 running Ubuntu 16.04 in 0.543 seconds which is well within the control frequency. The same Q_{LQR} and R_{LQR} matrices were used as in [10] for glucose and lactate state costs and input costs, respectively. These values are shown below for Q_{LQR}:

$$\begin{matrix}{G}_{IC , in} : q = 50 & {L}_{IC , in} : q = 50 \\ {G}_{IC , cell} ( l ) : q = {10}^{5} & {L}_{IC , cell} ( l ) : q = {10}^{5} \\ {G}_{out} : q = 5 \times {10}^{5} & {L}_{out} : q = 5 \times {10}^{5} \\ {G}_{EC , in} : q = {10}^{5} & {L}_{EC , in} : q = {10}^{5}\end{matrix} \qquad ( 14 )$$

$$and {R}_{LQR} :$$

$${R}_{LQR} = [ \begin{matrix}10 & 0 & 0 & 0 & 0 \\ 0 & 10 & 0 & 0 & 0 \\ 0 & 0 & 1 & 0 & 0 \\ 0 & 0 & 0 & 0.01 & 0 \\ 0 & 0 & 0 & 0 & 0.01\end{matrix} ] \qquad ( 15 )$$

However, with the addition of the discretized parameters θ, additional matrix terms were defined as follows in the LQR optimization problem for cost J:

$$J = {\int }_{0}^{∞} ( [ {X}^{T} {θ}^{T} {u}^{T} ] [ \begin{matrix}{Q}_{LQR} & 0 & 0 \\ 0 & {Q}_{θ} & {N}_{LQR} \\ 0 & {N}_{LQR}^{T} & {R}_{LQR}\end{matrix} ] [ \begin{matrix}X \\ θ \\ u\end{matrix} ] ) dt \qquad ( 16 )$$

The Q_{θ} matrix is 0 except for the nᵥ terms, which are equal to 3×10⁻⁷. The N_{LQR} matrix is 0 except for the nᵥ× [symbol] _{IC,m}, [symbol] _{EC,m} terms, which are equal to −n_{seeded}. These two terms combine together to drive the cost down to 0 only if the control effort u increases exponentially to match the exponentially increasing nᵥ during the expansion. This addition removes the need for a feedforward controller. The full exemplary control architecture is shown in FIG. 20.

### Results

Simulation Results. Each system started with the same initial cell number, glucose and lactate concentrations, and flow rates. Simulations ended once the 80% confluence limit was reached (˜6 days). The exemplary bioreactor physical parameters, cell behavior parameters, and the initial conditions are summarized in Table I and match the initialized parameters of the model validation expansion shown in FIG. 23.

TABLE I
Simulation Parameters

| | |
|---|---|
| S_{c}[μm²] [15] | ~1000 |
| S_{f}[m²] | 0.30 |
| C_{g}⁰[1/hr] | ~0.021 |
| G_{c}[mg/(cell * hr)] | ~−2 × 10⁻⁸ |
| Lₚ(mmol/(cell * hr)] | ~2.8 × 10⁻¹⁰ |
| n_{seeded} | 3 × 10⁶ cells |
| Media glucose, lactate | 1.15 g/L, 0.0 g/L |
| Concentrate glucose, lactate | ~1.8 g/L, 0.0 g/L |
| [symbol] _{IC, loop}, [symbol] _{EC, loop} | 3.2 mL/min, 4.0 mL/min |

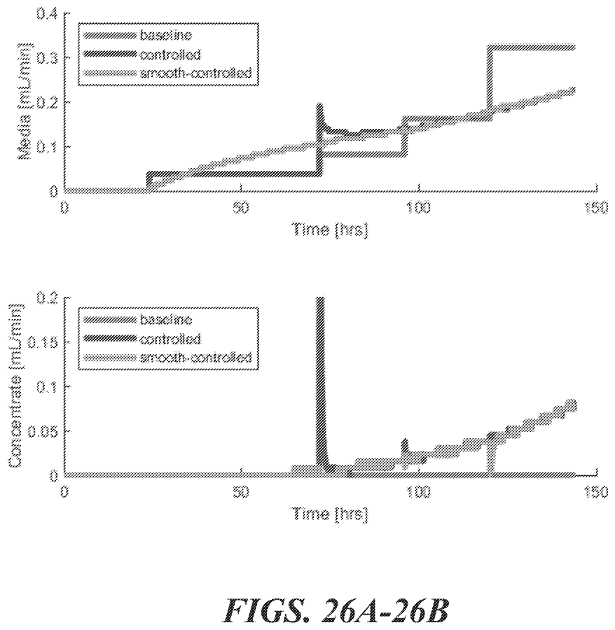

The simulation of the digital twin model and feedback controller was developed in MATLAB. The EKF observer and LQR controller were also simulated using the sensed states, sensing sampling rates (once per day), sensor noise, and controller update rates (1 Hz). The simulation results are shown in Table II, and the input flowrates are shown in FIG. 26A-26B.

TABLE II
Simulation Results

| | | | Smooth- |
|---|---|---|---|
| | Baseline | Controlled | Controlled |
| S-MSE Glucose [g/L] | 0.016 | 0.010 | 0.009 |
| S-MSE Lactate [g/L] | 0.038 | 0.032 | 0.029 |
| O-MSE Gluc./Lac. [g/L] | 3 × 10⁻⁵ | 1 × 10⁻⁵ | 1.5 × 10⁻⁵ |
| Media [L] | 0.920 | 0.812 | 0.823 |
| Concentrate [L] | 0.000 | 0.152 | 0.135 |
| # Lactate Spikes | 3 | 1 | 0 |

TABLE III
Bioreactor Expansion Results

| | Expansion 1 | | Expansion 2 | |
|---|---|---|---|---|
| | Glucose | Lactate | Glucose | Lactate |
| S-MSE [g/L] | 0.005 | 0.031 | 0.005 | 0.013 |
| O-MSE [g/L] | 3.9 × 10⁻² | 1.2 × 10⁻³ | 2.5 × 10⁻³ | 9.6 × 10⁻⁴ |

| | | |
|---|---|---|
| Media [L] | 0.660 | 0.380 |
| Conc. [L] | 0.150 | 0.190 |
| # Spikes | 1 | 0 |
| Cell Yield | 5.08 × 10⁷ | 4.91 × 10⁷ |

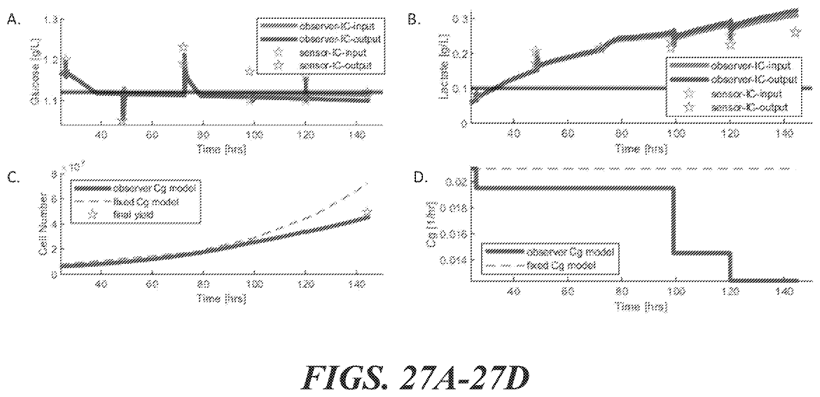

Expansion Results. In addition to the model validation controlled bioreactor expansion (Expansion 1), a second bioreactor expansion (Expansion 2) was performed using the same initialized parameters shown in Table I and the same cell/reagent lots in the model validation expansion (FIG. 23). This second expansion implemented an updated control architecture, parameter estimation model, smooth controller, and glucose and lactate setpoints (1.12 g/L and 0.1 g/L respectively) to eliminate error discontinuities. The second bioreactor expansion results are shown in FIG. 27A-27D for glucose (FIG. 27A), lactate (FIG. 27B), cell number (FIG. 27C), and cell growth rate (FIG. 27D). Controller and observer performance metrics as well as media usage and total cell yield are shown in Table III comparing the two bioreactor expansions. The observer cell yield in FIG. 27 (4.6×10⁷) is also within 6% of the true cell yield (4.91×10⁷).

The simulation results show that the controlled case improved glucose and lactate setpoint MSE compared to the baseline, while the smooth-controlled case improved setpoint MSE compared to both the controlled and baseline. Moreover, the large flowrate spike in the controlled case, reflected in the sudden nutrient spike in the model validation expansion (FIG. 23), was eliminated in the smooth-controlled case, which can maintain a more steady cell growth rate. Comparing the bioreactor expansion results, expansion 2 improved lactate setpoint MSE (58% reduction) while also significantly reducing observer MSE (36% for glucose and 20% for lactate). The improved observer MSE especially implies that the cell growth rate and cell number estimation were accurate. Although expansion 2 did not maintain a higher cell growth rate or result in a higher cell yield than expansion 1, the accurate parameter estimation resulted in less control effort and a large increase in cell yield per mL fed media (6.3×10⁴ cells/mL compared to 8.6×10⁴ cells/mL).

The impact of specific flow rates and flow rate trajectories on cell growth rate to enable direct regulation can be measured. The impact of different glucose and lactate reference profiles on cell growth rates can also be studied.

Model predictive control (MPC) specifically has been used for cell growth prediction in small-scale static cultures [8] and would improve on current estimation models, which cannot predict parameter trajectories. Post-expansion cell quality assays can be leveraged for MSC functions such as IDO suppression [16], macrophage M1 suppression [17], and T-cell co-culture [18] to determine key metabolites and cytokines to add to the model. With this detailed metabolite and quality data incorporated into an MPC algorithm, it is possible to predict and regulate cell growth parameters and quality assay metrics directly.

### Example #5: Artificial Intelligence-Enabled Biomanufacturing of Cell Therapies

Background. As cell therapies emerge as a new class of transformative “living medicines”, their large-scale manufacturing and bioprocessing require significant optimization and new paradigms to be commercially viable. FDA-approved immunotherapies (e.g., chimeric antigen receptor (CAR)-T cells) and experimental cell therapies (e.g., Mesenchymal Stromal Cells (MSCs)) are in high demand for clinical use, although advancements of current biomanufacturing platforms are necessary to develop, scale up and supply high-quality therapeutic products. Bioreactors currently provide culture environments to expand the number of cells needed for therapeutic dosages for patients [1, 2]. Cell expansion involve supplying vital nutrients in media (e.g., glucose, cytokines), removal of waste (e.g., lactate) and monitoring the growth of cells throughout time [3], all of which can be efficiently and non-destructively enabled by sensing, feedback controls, modeling, imaging, automation, and machine learning. However, this proposed “next-generation” bioreactor has yet to be developed due to several critical challenges involving each of the integrated components. Herein, this example describes a design that addresses some of the current biomanufacturing challenges, which will provide the groundwork for advanced biomanufacturing.

Suspension culture is an excellent choice for scaling up cell expansion because it can ensure the homogeneity of cells and nutrient distribution in the culture vessel, ensuring better process performance. Microcarriers are often associated with suspension culture for anchorage-dependent cell types that require adherence to a surface for cell proliferation (e.g., MSCs) or interaction with beads for functionalization (e.g., activated T cells). Although stirred tank bioreactors have been the method of choice for suspension culture in the biopharmaceutical production industry, cell therapy biomanufacturing systems are complex by the specifications of each therapeutic cell type to support cell quality and quantity. PBS Mini® vertical wheel bioreactors are suspension culture-based platforms that provide a uniform, low-shear fluid mixing environment for efficient particle suspension. The agitation mechanics provided by the vertical impeller and the shape of the vessel allows for a better homogenization of the microcarriers with low power input compared to stirred tank bioreactors with impellers. The low power and, consequently, low hydrodynamic shear makes this bioreactor specifically a great candidate for a culture of shear-sensitive, anchorage-dependent cells like MSCs on microcarriers [4].

However, given the fact that PBS Mini® has emerged recently as a bioreactor for cell therapy manufacturing, there has been a limited amount of optimization on the cell expansion process using this platform. Most of the studies published involve batch or fed-batch type of process, which could lead to nutrient depletion and toxic metabolite build-up. Manual feeding and extensive sampling can also introduce disturbances to the culture process, which negatively affect the manufactured product. Few in-line monitoring techniques to monitor critical nutrients and metabolites in media (e.g., glucose and lactate), and cell growth or cell health have been demonstrated, although highly desired to advance bioprocessing. Without in-line and non-destructive techniques, the current limitations result in suboptimal expansion levels, low product quality and/or quantity, and batch-to-batch variations.

Herein, the study introduced a feedback-controlled vertical wheel-based bioreactor with real-time imaging capabilities for monitoring cell density throughout the expansion (i.e., biomanufacturing) of MSCs and T cells. The experiment integrated modeling and feedback controls that interfaces commercially available in-line flow through sensors for glucose and lactate measurements with a designed interface composed of a transmitter and controller to update the feed flow rate and waste flow rate to replenish fresh media. The study also developed interferometric sensors for capturing dosed concentrations of specific proteins (e.g., IL-2, IL-15) that will further enable monitoring and feedback controls for T cell-specific biomanufacturing. A circulation loop extends out of the bioreactor, where cells and microcarriers can be circulated into a flow cell chamber and imaged by an optical fiber-based imaging device for non-destructive image-based cell density monitoring. Additionally, the example describes an automated sampling platform that replaces manual tasks of sampling, and demonstrated a high utility for ease of integration into biomanufacturing platforms.

### Methods and Materials.

Vertical wheel bioreactor, cells on microcarriers. Microcarriers provide a large surface area for MSCs to be culture expanded in a suspension environment. Microcarriers are commercially available in a range of sizes and surface coatings to enable cells to bind and proliferate to achieve a high yield of cells as the biomanufactured product [5]. Cultispher® is a macro-porous gelatin-based microcarrier that has been evaluated and deemed suitable for MSC cultivation [3]. Cultispher® was chosen for this study because the porosity of the beads makes them amenable for fiber optics-based imaging and permits image-based cell density measurements throughout the expansion of MSCs or T cells. Additionally, the example developed a robust T cell expansion system that modifies Cultispher® microcarriers in order to allow for T cell activation. The study showed that this system, termed Degradable Microscaffolds (DMS), improved cell yield and key phenotypes of cells compared to standard culture systems. The DMS system can drive higher density culture of T cells functionalized to microcarriers.

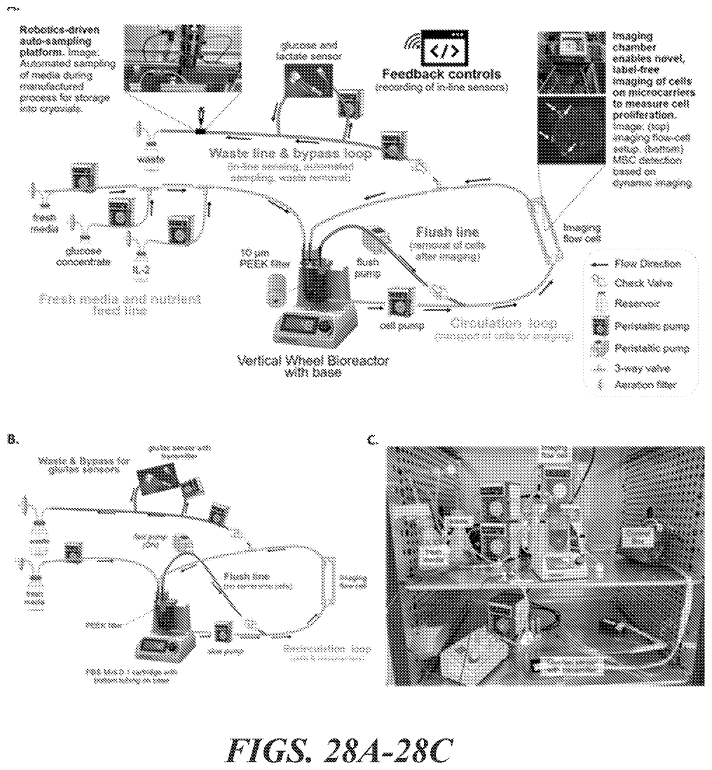

For a typical experimental setup, the PBS Mini® bioreactor would have an inlet port on the cap with flow rate-controlled media being fed in and an outlet port with a filter that flows the same flow rate as the feed but retain cells and microcarriers in the vessel. A circulation loop extends from the bottom and goes back into the cap that can circulate cells and microcarriers in the flow cell for imaging. Lastly, a flush line goes into the circulation loop to use filtered media to flush cells and microcarriers back in the vessel when not being imaged (FIG. 28A).

Feedback controls and hydraulics modeling. Given this experimental setup, hydraulic modeling techniques can be leveraged to model glucose and lactate concentrations within the reactor. This non-linear model assumes that all the significant volumes (bioreactor, sensor, and tubing) are continuously mixed and therefore have uniform substrate concentrations [6]. Given the PBS Mini® uses a vertical wheel to agitate the cells and mix the media, this assumption is applied. The designed model then continuously computes glucose and lactate concentrations in each of the volumes above given input volumetric flow rates of media and dosing solutions.

This model tracks concentrations in multiple volumes, but only the sensor volume can actually be measured, which does not accurately inform concentrations within the bioreactor. With the addition of a nonlinear Extended Kalman Observer [7], the forward simulated model concentrations can be combined with the intermittent or continuous measurement at the sensor volume to optimally track concentrations in all the volumes.

With the full state information, a Linear Quadratic Regulator was designed to control the input media and dosing flowrates [7]. The controller can optimally regulate to any desired glucose and lactate trajectory, limited by the amount of media and dosing solutions provided. For this application, it was desired to hold glucose constant and keep lactate low without allowing for large spikes in the concentrations in order to keep a consistent cell growth rate.

Sensors. Glucose and lactate are commonly measured manually once per day and provide an indication of cell growth rate as well as culture success. Commercial flow-through glucose and lactate oxidative sensors have been integrated into the overall setup to automate the sampling process and provide continuous feedback to the controllers.

GTRI has developed a field-tested, miniaturized optical sensor capable of detecting a wide variety of chemical and biological species. Central to the device is a planar optical waveguide with an evanescent field that is sensitive to minute changes in the volume immediately above the surface. Optically combining a guided sensing beam with a reference beam generates an interference fringe pattern whose phase changes in proportion to the index of refraction differences between the two arms of the interferometer. Applying a chemically selective film over the sensing arm of the interferometer provides the basis for the chemical sensor.

Imaging techniques. Cell growth and density were visually monitored by label-free, non-invasive imaging of the microcarriers flowing through the flow cell. Imaging of the 200-300 um thick microcarriers was enabled by quantitative Oblique Back Illumination (qOBM) [8-10], a label-free microscopy technique that enables 3D quantitative phase images with an epi-illumination configuration that is well suited for imaging of thick, highly-scattering objects such as microcarriers. QOBM has the geometry of a conventional inverted microscope with a modified illumination scheme. The sample is illuminated from below (epi-illumination) by four LEDs (720 nm) coupled into 1 mm optical fibers, positioned 90 degrees around the microscope objective (Nikon CFI S Plan Fluor ELWD 40×). During the imaging process, four raw captures were acquired, one for each of the four LEDs. When the sample was illuminated, light underwent multiple scattering events during which some of the photons are redirected towards the objective and effectively produced a virtual light source within the sample. An additional ˜2 mm thick layer of polydimethylsiloxane was placed above the flow cell to act as a diffuser which ensures sufficient backscattered light illuminates the focal plane and that the scattered light can be accurately modeled, which is significant for qOBM. Each raw capture carries unique angular information about the refractive index changes of the sample. The final qOBM image reconstruction includes the four captures and a model of the optical transfer function of the system to obtain the quantitative phase images [8].

While the microcarriers' thickness did not permit the use of conventional transmission-based, label-free cell imaging techniques (e.g., phase contrast, DIC, or QPI), qOBM delivers unique tomographic, quantitative phase contrast of cells attached to the microcarriers, as well as free-floating cells in real-time, non-invasively.

Machine Learning. The platform generated a large amount of sensor data (e.g., optical and chemical sensors) as well as a history of control actions. Machine Learning algorithms developed for the platform aim to characterize the cells based on this rich time-series data in order to provide inputs to the control system as well as predict current and future cell quality during the expansion process.

The problem poses a challenge to Machine Learning algorithms. The fundamental difficulty is the complexity and variability of the underlying biological process, which needs to be modeled in order for Machine Learning system to make correct predictions. However, the ability to generate a large amount of full data sequences is very limited, as each expansion run is expensive and time-consuming. As a result, there was a large amount of sensor data for each experiment but very few experimental runs with the corresponding outcome (i.e., success/fail or cell quality metric).

The model devised for the platform included a set of LSTM-based feature extractors for the available data sources (glucose and lactate sensors; imaging; control actions), each having a different frequency. The resulting embeddings were fused together and fed to a “main loop” LSTM model that had a larger time step. The main LSTM modeled the expansion process by predicting the next control inputs and expected sensor readings at each step, as well as the overall outcome based on its final state. The training of the model is possible by comparing those predictions to the ground truth in consecutive time steps. This pairwise approach allows for achieving a denser supervision during training despite a lack of human-provided labels and a limited number of final outcomes.

Automation. In order to implement the feedback control and sensing discussed previously, automated means of actuating the system are required in order to act on the information gained from the sensors and control models. There is a long list of system parameters whose control will be necessary, which include the volumetric flow rates of the input media and nutrients, output waste, imaging recirculation, and flushing loops. Also required is an actuation of the vertical wheel, automatic sampling system, and the imaging subsystem.

The integration of each of these automated actions (pumps, vertical wheel, sampling, imaging) will realize a number of important benefits for both the usability of the system and the viability of the cells cultured in it. The primary benefit of this completely integrated and automated system is the ability to monitor the health and growth of the cells throughout the entire expansion autonomously. Automation of tasks and feedback controls enables a stabilized culture environment where levels of nutrients and waste are maintained, theoretically improving the quality of the manufactured cell therapy product. Moreover, automation eliminates the time, labor, and errors related to user-driven manual tasks.

Beyond the improvements to growth afforded by the automated feeding and sampling, automating the feeding and sampling process decreases the chance of culture death through contamination or researcher error. Carefully controlling the feeding rates automatically removes that burden from the researcher, avoiding the situation where a delayed or missed dose would result in the loss of cells. Automatic dosing and sampling also maintain a closed system, eliminating a potential entry point for contaminants.

### Technical Results.

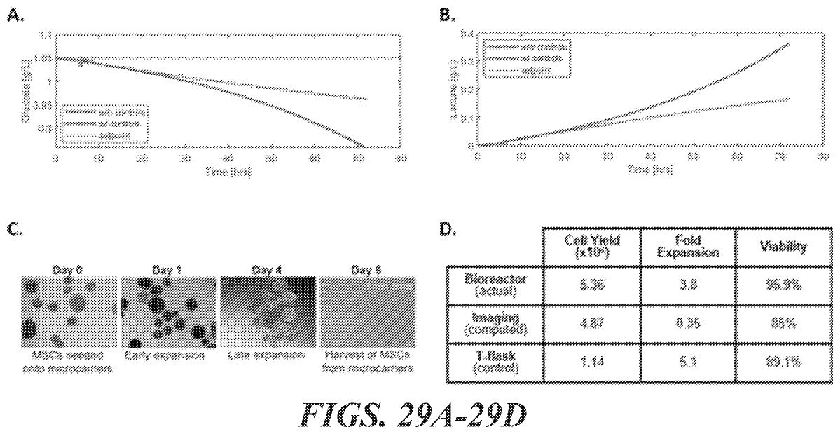

The platform's assembly was shown to incorporate a fresh media loop to introduce fresh media and is capable of introducing specific proteins or nutrients of interest into the bioreactor. The bioreactor was able to culture MSCs on microcarriers in turbulent flow provided by the vertical wheel. A circulation loop was able to transport cells on microcarriers from the bioreactor unit into a flow cell that enabled imaging of the cells by advanced microscopy techniques or camera and then return the cells on microcarriers back to the bioreactor for continued expansion (FIG. 28B-28C). These results have demonstrated that modeling by simulations of glucose and lactate levels (FIG. 29A-29B) achieves the necessary controls when integrated into the bioreactor platform. Further, preliminary results show the feasibility of the methodology for measuring cell yield/density and viability captured by imaging (computed) and actual expansion of MSCs on microcarriers in the bioreactor platform assembly shown in FIG. 28C (FIG. 29C-29D).

Imaging of the microcarriers through the flow cells was performed to determine the cell density of expanding cells. Using qOBM, MSCs, T cells, and microcarriers were successfully imaged. To achieve this, each microcarrier was imaged with qOBM at 5 Hz for a short period of time (˜1 min), capturing a time-lapse of images at a single region of interest. The time lapses were then analyzed to generate dynamic maps that easily differentiate cells from the microcarrier, which possesses a complex structure that can otherwise obscure cells. Microscope and camera images were further evaluated using machine learning methods to determine cell density and dynamic activity (related to metabolism and cell viability), which will theoretically inform the status of the cells in the bioreactor users of the platform.

The outgoing loop, which enabled in-line sensing, automated sampling, and waste removal, heavily relied on a suitable filter that would allow for media, and not cells or microcarriers, to be pumped out of the bioreactor unit. Due to the availability of commercial filters, size, and material constraints, specifications for use, identifying a filter for the outgoing loop posed a great challenge, and several filters were tested. Fortunately, progress has been made in developing an engineered filtering technique to enable media removal using the outgoing loop. Further iterations will tune the engineered filter for optimal use in the working platform.

Commercially available in-line sensors were used to measure changing glucose and lactate levels that were fed into modeling and feedback controls. Experience using these sensors has enabled troubleshooting and identified shortcomings of the sensors that may be due to biofouling or diminished sensitivity to analytes. Currently, alternative in-line sensors are planned for use in upcoming iterations. Novel sensor development for measuring dosed proteins has been accomplished and shown the range of sensitivity based on concentration. Integration of the novel sensors will be performed in upcoming iterations of the working platform and further enable the capabilities of modeling and feedback controls related to protein dosing. The automated sampling platform has been independently shown to perform to the specifications of the users for sampling media from bioreactors. Sterilization testing is planned to demonstrate the broad integration of the automated sampling platform that will enable automated sample collection and sample transfer into plates or reservoirs for various downstream off-line analytics of the media.

Summary. This first proof-concept next-generation bioreactor was developed and tested in several stages to test the utility of integrated components, capture data for controls, imaging, and ML, and determine feasible culture conditions for cells on microcarriers. The experience, knowledge gained, and data captured to date are necessary to understand the future directions for the proposed next-generation bioreactor. Although much progress has been made to lay the groundwork for a next-generation bioreactor platform, considerable efforts to tune the working components and optimize the components or techniques in development are necessary to achieve the goals for all integrated capabilities of the bioreactor throughout cell expansion and full-scale use.

### Additional Embodiments

Bioreactor performance. The filter is key to the flush line since cell-free media is required to flow the carriers out of the imaging cell. It is also key to the waste line to ensure that cells are not discarded to waste. Preliminary testing with the filter showed that cells and carriers easily clogged the filter, which led to increased pressure and the inability to pull media. Without a working filter, neither controls nor imaging can be safely implemented. A promising solution being explored is to include an additional filter with a much larger surface area closer to the vertical wheel, thereby reducing pressure at the output line and carrier/cell buildup on the filter. Novel sensing integration. Glucose and lactate commercial sensors have been integrated with the control architecture described prior. The novel sensors are currently being evaluated offline for accuracy may incorporate continuous feedback from the novel sensors for specific molecules in the media (such as IL-2), which can then be tracked in the controller and used for additional dosing medias.

Imaging integration. The imaging module has been well characterized and successfully tested using a flow-cell to enable facile access to the microcarriers and cells within. While this is indeed a viable configuration to monitor cells non-invasively, future work will focus on developing a more seamless integration between the imaging module and bioreactor. For example, suspended cells and adherent cells attached to microcarriers can be monitored directly inside the bioreactor using the same qOBM technology. This can be achieved with a few modifications to the optical module and/or the bioreactor.

ML development/analysis. The proof-of-concept Machine Learning model, following evaluation on the experimental data, will likely need significant refinement. Likely, a main focus would be to develop techniques addressing the scarcity of labels, data imbalance, covariate shift (due to few experimental observations), and, more broadly, promote the generalizability of the model. The latter point is particularly important given multiple potential cell types and sources and the heterogeneous sensor setup. The incorporation of first-principles modeling and novel sensor fusion techniques will need to be explored.

Automation. The PBS Mini bioreactor vessel was not designed with automation in mind. As such it has required significant modifications in order to facilitate automated in-line sensing, imaging, and control. Individual modifications, including control of the bioreactor wheel, addition of recirculation tubing, and automation of a microscope stage, have all been developed separately. However, the integration of these components into a complete automated system have yet to be completed. Future work on this front will include the combination of individual automated components into a full closed-loop system, requiring both hardware and software integration. Once a unified system has been developed, additional tests will be required to validate the functionality of the system and prove its benefit to cell quality over traditional manually controlled processes. In addition, an intuitive user interface will be developed, which could enable researchers to configure culture parameters and indicate desired CQAs.

### Discussion

The FDA Hollow Fiber Controlled Bioreactor platform is a small research-scale bioreactor platform consisting of a Fibercell hollow fiber cartridge augmented with automated flow control, inline sensors for glucose, lactate, and cell density, and an automated sampling platform for collecting samples for offline analysis. This platform is designed to function as a scaled-down version of the Terumo Quantum bioreactor for cheaper test batches used to tune the culture process. In addition, this system introduces inline sensors and a novel feedback controller, which is used to dynamically monitor and control what is previously an unregulated environment. Current practice involves the use of fixed “recipes” for cell density, feeding, and harvest.

Other current state-of-the-art bioreactors, such as the Terumo Quantum, rely on offline sampling and manual adjustment of feed rates in their expansion protocols. Utilizing a fixed recipe for culturing is unable to account for variabilities in the expansion process, as is often the case with unique donor cells. Moreover, the optimization of large-scale bioreactors is limited by resources and cost. Therefore, a medium-scale bioreactor system with the capabilities of automatic sampling, in-line sensing and control is highly desired. Described herein is a designed medium-scale hollow fiber (MSHF) bioreactor with integrated in-line sensors that inform mathematical models and feedback controls and an automated sampling platform making a semi-autonomous biomanufacturing platform for MSCs.

The exemplary system employs a proof-of-concept design that can serve as the groundwork for a next-generation bioreactor for cell therapy products that permits plug-and-play integration of analytical technologies as biological discoveries, and engineering innovations craft the biomanufacturing landscape for cell therapies.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entirety and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organisms, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document are hereby incorporated by reference in their entirety herein.

### Examples 1-3

[1] https://ieeexplore.ieee.org/document/9319723—This feedback controller was published by one of the researchers on this project and its technology is in use in the control of this system.
[2] Hanley, P. J., et al., Efficient manufacturing of therapeutic mesenchymal stromal cells with the use of the Quantum Cell Expansion System. Cytotherapy, 2014. 16(8): p. 1048-58.
[3] Frank, N. D., et al., Evaluation of reagents used to coat the hollow-fiber bioreactor membrane of the
[4] Quantum® Cell Expansion System for the culture of human mesenchymal stem cells. Mater Sci Eng C Mater Biol Appl, 2019. 96: p. 77-85.
[5] Barckhausen, C., et al., GMP-Compliant Expansion of Clinical-Grade Human Mesenchymal Stromal Stem Cells Using a Closed Hollow Fiber Bioreactor. Methods Mol Biol, 2016. 1416: p. 389-412.
[6] Mizukami, A., et al., A Fully-Closed and Automated Hollow Fiber Bioreactor for Clinical-Grade Manufacturing of Human Mesenchymal Stem/Stromal Cells. Stem Cell Rev Rep, 2018. 14(1): p. 141-143.
[7] Nold, P., et al., Good manufacturing practice-compliant animal-free expansion of human bone marrow derived mesenchymal stroma cells in a closed hollow-fiber-based bioreactor. Biochem Biophys Res Commun, 2013. 430(1): p. 325-30.
[8] Vymetalova, L., et al., Large-Scale Automated Hollow-Fiber Bioreactor Expansion of Umbilical Cord-Derived Human Mesenchymal Stromal Cells for Neurological Disorders. Neurochem Res, 2020. 45(1): p. 204-214.
[9] Hanley, Patrick J et al. “Efficient manufacturing of therapeutic mesenchymal stromal cells with the use of the Quantum Cell Expansion System.” Cytotherapy vol. 16,8(2014): 1048-58. doi: 10.1016/j.jcyt.2014.01.417.
[10] Moïra François, Raphaë11e Romieu-Mourez, Mengyang Li, Jacques Galipeau, Human MSC Suppression Correlates With Cytokine Induction of Indoleamine 2,3-Dioxygenase and Bystander M2 Macrophage Differentiation, Molecular Therapy, 20, 1(2012): 187-95, ISSN 1525-0016.
[11] Hanley, Patrick J et al. “Efficient manufacturing of therapeutic mesenchymal stromal cells with the use of the Quantum Cell Expansion System.” Cytotherapy vol. 16,8(2014): 1048-58. doi: 10.1016/j.jcyt.2014.01.417.

### Example 4

[1] K. Le Blanc, I. Rasmusson, B. Sundberg, C. Götherström, M. Hassan, M. Uzunel, and O. Ringden, “Treatment of severe acute graft-versushost disease with third party haploidentical mesenchymal stem cells,” Lancet, vol. 363, pp. 1439-1441 May 2004.
[2] I. Molendijk, B. A. Bonsing, H. Roelofs, K. C. M. J. Peeters, M. N. J. M. Wasser, G. Dijkstra, C. J. van der Woude, M. Duijvestein, R. A. Veenendaal, J.-J. Zwaginga, H. W. Verspaget, W. E. Fibbe, A. E. van der Meulen-de Jong, and D. W. Hommes, “Allogeneic bone marrowderived mesenchymal stromal cells promote healing of refractory perianal fistulas in patients with crohn's disease,” Gastroenterology, vol. 149, pp. 918-27.e6, October 2015.
[3] J. Panés, D. García-Olmo, G. Van Assche, J. F. Colombel, W. Reinisch, D. C. Baumgart, A. Dignass, M. Nachury, M. Ferrante, L. Kazemi-Shirazi, J. C. Grimaud, F. de la Portilla, E. Goldin, M. P. Richard, A. Leselbaum, S. Danese, and ADMIRE CD Study Group Collaborators, “Expanded allogeneic adipose-derived mesenchymal stem cells (cx601) for complex perianal fistulas in crohn's disease: a phase 3 randomised, double-blind controlled trial,” Lancet, vol. 388, pp. 1281-1290 September 2016.
[4] A. Mizukami and K. Swiech, “Mesenchymal stromal cells: From discovery to manufacturing and commercialization,” Stem Cells Int., vol. 2018, p. 4083921, April 2018.
[5] M. Kabat, I. Bobkov, S. Kumar, and M. Grumet, “Trends in mesenchymal stem cell clinical trials 2004-2018: Is efficacy optimal in a narrow dose range?,” Stem Cells Transl. Med., vol. 9, pp. 17-27, January 2020.
[6] C. Barckhausen, B. Rice, S. Baila, L. Sensebé, H. Schrezenmeier, P. Nold, H. Hackstein, and M. T. Rojewski, “GMP-Compliant Expansion of Clinical-Grade Human Mesenchymal Stromal/Stem Cells Using a Closed Hollow Fiber Bioreactor,” Methods in Molecular Biology (Clifton, N.J.), vol. 1416, pp. 389-412, 2016.
[7] A. L. Russell, R. C. Lefavor, and A. C. Zubair, “Characterization and cost-benefit analysis of automated bioreactor-expanded mesenchymal stem cells for clinical applications,” Transfusion, vol. 58, pp. 2374-2382 October 2018.
[8] K. Van Beylen, A. Youssef, A. Peña Fernandez, T. Lambrechts, I. Papantoniou, and J.-M. Aerts, “Lactate-based model predictive control strategy of cell growth for cell therapy applications,” Bioengineering (Basel), vol. 7, p. 78, July 2020.
[9] R. B. Rice, R. Peters, B. Vang, M. Brecheisen, T. Startz, B. Nankervis, D. Windmiller, D. Antwiler, N. Givens, and M. E. Janssens, “Glucose and lactate measurements as a predictor of the human mesenchymal stem cell number in the quantum cell expansion system,” 2012.
[10] B. Kanwar, S. Balakirsky, and A. Mazumdar, “Modeling and Controller Design for Enhanced Hollow-fiber Bioreactor Performance,” IEEE Control Systems Letters, pp. 1-1, 2021.
[11] I. A. Udugama, P. C. Lopez, C. L. Gargalo, X. Li, C. Bayer, and K. V. Gernaey, “Digital twin in biomanufacturing: challenges and opportunities towards its implementation,” Systems Microbiology and Biomanufacturing, 2021.
[12] D. Salzig, J. Leber, K. Merkewitz, M. C. Lange, N. Köster, and P. Czermak, “Attachment, Growth, and Detachment of Human Mesenchymal Stem Cells in a Chemically Defined Medium,” February 2016.
[13] F. A. M. Abo-Aziza and Z. A A, “The impact of confluence on bone marrow mesenchymal stem (BMMSC) proliferation and osteogenic differentiation,” Int. J. Hematol. Oncol. Stem Cell Res., vol. 11, pp. 121-132, April 2017.
[14] X. Yuan, T. M. Logan, and T. Ma, “Metabolism in Human Mesenchymal Stromal Cells: A Missing Link Between hMSC Biomanufacturing and Therapy?,” Frontiers in Immunology, vol. 10, May 2019.
[15] J. R. Smith, K. Pfeifer, F. Petry, N. Powell, J. Delzeit, and M. L. Weiss, “Standardizing umbilical cord mesenchymal stromal cells for translation to clinical use: Selection of GMP-compliant medium and a simplified isolation method,” Stem Cells Int., vol. 2016, p. 6810980, February 2016.
[16] O. DelaRosa, E. Lombardo, A. Beraza, P. Mancheño-Corvo, C. Ramirez, R. Menta, L. Rico, E. Camarillo, L. García, J. L. Abad, C. Trigueros, M. Delgado, and D. Büscher, “Requirement of IFN-gamma-mediated indoleamine 2,3-dioxygenase expression in the modulation of lymphocyte proliferation by human adipose-derived stem cells,” Tissue Eng. Part A, vol. 15, pp. 2795-286 October 2009.
[17] A. B. Vasandan, S. Jahnavi, C. Shashank, P. Prasad, A. Kumar, and S. J. Prasanna, “Human mesenchymal stem cells program macrophage plasticity by altering their metabolic status via a PGE2-dependent mechanism,” Sci. Rep., vol. 6, p. 38308, December 2016.
[18] D. D. Bloom, J. M. Centanni, N. Bhatia, C. A. Emler, D. Drier, G. E. Leverson, D. H. McKenna, Jr, A. P. Gee, R. Lindblad, D. J. Hei, and P. Hematti, “A reproducible immunopotency assay to measure mesenchymal stromal cell-mediated t-cell suppression,” Cytotherapy, vol. 17, pp. 140-151, February 2015.

### Example 5

[1] Haddock, R., Lin-Gibson, S., Lumelsky, N., McFarland, R., Roy, K., Saha, K., Zhang, J., Zylberberg, C., Manufacturing Cell Therapies: The Paradigm Shift in Health Care of This Century. 2017, National Academy of Medicine Perspectives.
[2] Riviere, I. and K. Roy, Perspectives on Manufacturing of High-Quality Cell Therapies. Mol Ther, 2017. 25(5): p. 1067-1068.
[3] Rafiq, Q. A., et al., Systematic microcarrier screening and agitated culture conditions improves human mesenchymal stem cell yield in bioreactors. Biotechnol J, 2016. 11(4): p. 473-86.
[4] Sousa, M. F., et al., Production of oncolytic adenovirus and human mesenchymal stem cells in a single-use, Vertical-Wheel bioreactor system: Impact of bioreactor design on performance of microcarrier-based cell culture processes. Biotechnol Prog, 2015. 31(6): p. 1600-12.
[5] Tsai, A. C., et al., Influence of Microenvironment on Mesenchymal Stem Cell Therapeutic Potency: From Planar Culture to Microcarriers. Front Bioeng Biotechnol, 2020. 8: p. 640.
[6] Rajinikanth, V. and K. Latha, Tuning and Retuning of PID Controller for Unstable Systems Using Evolutionary Algorithm. ISRN Chemical Engineering, 2012. 2012: p. 1-11.
[7] Kanwar, B., S. Balakirsky, and A. Mazumdar, Modeling and Controller Design for Enhanced Hollow-Fiber Bioreactor Performance. IEEE Control Systems Letters, 2022. 6: p. 115-120.
[8] Ledwig, P. and F. E. Robles, Epi-mode tomographic quantitative phase imaging in thick scattering samples. Biomedical Optics Express, 2019. 10(7): p. 3605-3621.
[9] Ledwig, P. and F. E. Robles, Quantitative 3D refractive index tomography of opaque samples in epi-mode. Optica, 2021. 8(1): p. 6-14.
[10] Costa, P. C., et al., Towards in-vivo label-free detection of brain tumor margins with epi-illumination tomographic quantitative phase imaging. Biomed Opt Express, 2021. 12(3): p. 1621-1634.

1. A method using model-based control for determining process parameters in a bioreactor in real time (inline), the method comprising:
a) providing a bioreactor comprising a culture chamber, wherein said culture chamber comprises live cells in culture media;
b) providing two or more sensors for measuring two or more physical attributes of the culture media or the live cells therein;
c) receiving, via a controller, sensor data from the two or more sensors operatively disposed on, or in a flow pathway of, the culture chamber; and
d) determining, via the controller, in real time, values associated with and derived from at least two physical attributes of the culture media or live cells therein using a model employing the sensor data from the two or more sensors; and
e) adjusting, via the controller, according to a pre-defined control parameter, control of the culture media in the bioreactor using the determined values associated with and derived from physical attributes of the culture media or live cells therein.

2. The method of claim 1, wherein the live cells comprise immune cells, stem cells, stem-derived cells, or red blood cells.

3. The method of claim 2, wherein the immune cells comprise chimeric antigen receptor (CAR) T cells, natural killer cells (NKs), T-regulatory cells (T-regs), T cells, or tumor infiltrating lymphocytes (TILs).

4. The method of claim 2, wherein the stem cells comprise induced pluripotent stem cells (PSCs), mesenchymal stem cells (MSCs), or MSC- or PSC-derived cells.

5. The method of claim 1, wherein the bioreactor comprises a stirred-tank, airlift, hollow-fiber, or rotary cell culture system (RCCS).

6. The method of claim 5, wherein the bioreactor is a small-scale or medium-scale bioreactor.

7. The method of claim 1, wherein said physical attribute comprises a critical quality attribute (CQA) and/or a critical process parameter (CPP).

8. The method of claim 1, wherein the two or more different physical attributes comprise dissolved oxygen, pH, glucose level, lactate concentration, temperature, conductivity, NO/NOx, volatile organic compounds (VOCs), ozone, chlorine, reduction-oxidation (redox) potential, agitation, inward gas flow, outward gas flow, pressure, impedance, resistance, vessel weight, metabolite concentrations, cytokine or growth factor concentrations, capacitance, cell size, cell dynamics, distribution of cell size or dynamics, or optical density.

9. The method of claim 1, wherein the sensors provide data at discrete time intervals or continuously.

10. The method of claim 1, wherein the method further comprises the following step:
f) a feedback control which automatically adjusts one or more physical parameters of the culture media based on the physical attributes of the culture media or cells detected by the sensors.

11. The method of claim 10, wherein one or more physical parameters are adjusted by addition of a composition.

12. The method of claim 10, wherein amount or type of adjustment of the physical parameter is variable based on feedback received for that physical parameter.

13. The method of claim 1, wherein the culture chamber comprises a perfusion bioreactor, a stirred bioreactor, or a static bioreactor.

14. The method of claim 1, wherein the model is an artificial intelligence (AI) model.

15. The method of claim 1, wherein the model can predict or estimate culture growth rate, cell number, or other cell or culture characteristics based on measured parameters.

16. A system for generating an AI digital twin model for a commercial-scale bioreactor, the system comprising:
a) a bioreactor comprising a culture chamber, wherein said culture chamber comprises live cells in culture media;
b) two or more sensors for measuring two or more different physical attributes of the culture media or the live cells therein; and
c) a computing device communicatively coupled to the two or more sensors, the computing device comprising
i) a processor;
ii) a memory; and
iii) a communication module;
the processor communicatively coupled to the memory and the communication module; the processor configured to receive sensor data from the two or more sensors to train an AI model configured to determine values associated with and derived from physical attributes of the culture media or live cells therein using the sensor data from the two or more sensors, wherein the trained AI model is subsequently employed in controls of the commercial-scale bioreactor to determine values associated with and derived from physical attributes of the culture media or live cells therein using sensor data from sensors of the commercial-scale bioreactor.

17. (canceled)

18. (canceled)

19. (canceled)

20. The system of claim 16, wherein the bioreactor comprises a stirred-tank, airlift, hollow-fiber, or rotary cell culture system (RCCS), and is configured as a small-scale or medium-scale bioreactor.

21. (canceled)

22. The system of claim 16,
wherein said physical attribute comprises a critical quality attribute (CQA) and/or a critical process parameter (CPP), and
wherein the two or more different physical attributes comprise dissolved oxygen, pH, glucose level, temperature, conductivity, NO/NOx, volatile organic compounds (VOCs), ozone, chlorine, reduction-oxidation (redox) potential, agitation, inward gas flow, outward gas flow, pressure, vessel weight, metabolite concentrations, cytokine or growth factor concentrations, impedance, capacitance, resistance, or optical density.

23. (canceled)

24. (canceled)

25. The system of claim 16, further comprising a feedback control component configured to automatically adjusts one or more physical parameters of the culture media based on the physical attributes of the culture media detected by the sensors.

26. The system of claim 16, wherein the culture chamber comprises a perfusion bioreactor, a stirred bioreactor, or a static bioreactor.
